(19) United States
(12) Patent Application Publication
(10) Pub. No.: US 2026/0116925 A1
(43) Pub. Date: Apr. 30, 2026

(54) FORMULATION FOR WOUND HEALING

(71) Applicant: ARIZONA BOARD OF REGENTS ON BEHALF OF ARIZONA STATE UNIVERSITY, Scottsdale, AZ (US)

(72) Inventors: Jordan Yaron, Scottsdale, AZ (US); Kaushal Rege, Gilbert, AZ (US)

(21) Appl. No.: 18/844,561

(22) PCT Filed: Mar. 10, 2023
(86) PCT No.: PCT/US2023/015004

§ 371 Date: Sep. 6, 2024

Related U.S. Application Data
(60) Provisional application No. 63/318,722, filed on Mar. 10, 2022.

(51) Int. Cl. C07K 14/00

(52) U.S. Cl. CPC C07K 14/001

(57) ABSTRACT

Wound healing is a process by which these wounds on the skin of a subject heal and eventually close. When the injured surface is large, becomes infected, or in patients with poor healing capacity such as diabetics or the elderly or bedridden patients, then wound healing can be prolonged and lead to chronic ulceration and further complications with even limb loss or increased morbidity and mortality. This invention is directed to a topical formulation for promoting wound healing, and wound dressings and bandages comprising the same.

This application is a National Stage Entry of PCT Application No. PCT/US2023/015004, filed on Mar. 10, 2023, which is an International Application that claims priority from U.S. Provisional Application No. 63/318,722 filed on Mar. 10, 2022, the entire contents of each of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTEREST

This invention was made with government support under R01 AR074627 awarded by the National Institutes of Health. The government has certain rights in the invention.

### REFERENCE TO A SEQUENCE LISTING XML

In accordance with 37 C.F.R. § 1.831, the present specification makes reference to a Sequence Listing which has been submitted electronically in XML format. The entire contents of the Sequence Listing XML are incorporated herein by reference, with the intention that, upon publication (including issuance), this incorporated Sequence Listing will be inserted in the published document immediately before the claims. Said XML file, created on Apr. 21, 2025, is named 2952332-000010-US2_SL.xml and is 80,879 bytes in size.

### FIELD OF THE INVENTION

This invention is directed to a topical formulation for promoting wound healing, and wound dressings and bandages comprising the same.

### BACKGROUND OF THE INVENTION

Wounds in mammalian tissue result in tissue disruption and coagulation of the microvasculature at the wound face. Wound healing is a process by which these wounds on the skin of a subject heal and eventually close. Repair of such tissue represents an orderly, controlled cellular response to injury. Soft tissue wounds, regardless of size, heal in a similar manner. Tissue regrowth and repair are biologic systems wherein cellular proliferation and angiogenesis occur in the presence of an oxygen gradient. The sequential morphological and structural changes which occur during tissue repair have been characterized in great detail and have in some instances been quantified. When the injured surface is large, becomes infected, or in patients with poor healing capacity such as diabetics or the elderly or bedridden patients, then wound healing can be prolonged and lead to chronic ulceration and further complications with even limb loss or increased morbidity and mortality.

### SUMMARY OF THE INVENTION

Aspects of the invention are drawn towards a polypeptide comprising an amino acid sequence at least 80% identical to an amino acid sequence according to MHLFAEATTKFTLELYRQLRESDNNIFYSPISMMTALAMLQLGAKGNTEKQIEKVLQ FNETTKKTTEKSAHCHDEENVHEQFQKLMTQLNKSNDAYDLKAANSIYGAKGFPFV QTFLEDIKEYYQANVESLDFEHAAEESEKKINSWVESQTNGKIKDLFPNGSLNRSTIM VLVNAVYFKGQWNHKFDEKHTTEEKFWLNKNTSKPVQMMKQNIEFNFMFLEDVQ AKIVEIPYKGKELSMIVLLPVEINGLKQLEEQLTADKLLEWTRAENMHMTELYLSLP RFKVDEKYDLPIPLEHMGMVDAFDPQKADFSGMSSTQGLVVSKVLHKSFVEVNEEG TEAAAATGVEVSLTSAQIAEDFCCDHPFLFFIIHRKTNSILFFGRISSP (SEQ ID NO: 18); MNSLSEANTKFMFDLFQQFRKSKENNIFYSPISITSALGMVLLGAKDNTAQQIKKVLH FDQVTENTTGKAATYHVDRSGNVHHQFQKLLTEFNKSTDAYELKIANKLFGEKTYL FLQEYLDAIKKFYQTSVESVDFANAPEESRKKINSWVESQTNEKIKNLIPEGNIGSNTT LVLVNAIYFKGQWEKKENKEDTKEEKFWPNKNTYKSIQMMRQYTSFHFASLEDVQ AKVLEIPYKGKDLSMIVLLPNEIDGLQKLEEKLTAEKLMEWTSLQNMRETRVDLHLP RFKVEESYDLKDTLRTMGMVDIFNGDADLSGMTGSRGLVLSGVLHKAFVEVTEEGA EAAAATAVVGFGSSPTSTNEEFHCNHPFLFFIRQNKTNSILFYGRFSSP (SEQ ID NO: 19); MNSLSEANTKFMFDLFQQFRKSKENNIFYSPISITSALGMVLLGAKDNTAQQISKVLH FDQVTENTTEKAATYHVDRSGNVHHQFQKLLTEFNKSTDAYELKIANKLFGEKTYQ FLQEYLDAIKKFYQTSVESTDFANAPEESRKKINSWVESQTNEKIKNLFPDGTIGNDT TLVLVNAIYFKGQWENKFKKENTKEEKFWPNKNTYKSVQMMRQYNSFNFALLEDV QAKVLEIPYKGKDLSMIVLLPNEIDGLQKLEEKLTAEKLMEWTSLQNMRETCVDLHL PRFKMEESYDLKDTLRTMGMVNIFNGDADLSGMTWSHGLSVSKVLHKAFVEVTEE GVEAAAATAVVVVELSSPSTNEEFCCNHPFLFFIRQNKTNSILFYGRFSSP (SEQ ID NO: 20); or a fragment thereof.

In embodiments, the polypeptide is recombinantly produced.

In embodiments, the fragment comprises an amino acid sequence at least 80% identical to the amino acid sequence of GTEAAAATGVEVSLTSAQIA (SEQ ID NO: 8); GAEAAAATAVVGFGSSPTST (SEQ ID NO: 9); GVEAAAATAVVVVELSSPST (SEQ ID NO: 10).

In embodiments, the fragment comprises a truncation mutant. For example, the truncation mutant comprises an N-truncated mutant or a C-truncated mutant. For example, the truncation mutant comprises a reactive center loop truncation mutant.

In embodiments, positions 71-76 of the polypeptide according to SEQ ID NO: 5 are replaced with TYHVDRS (SEQ ID NO: 11) or YHVDRS (SEQ ID NO: 12), wherein positions 346-356 according to SEQ ID NO: 5 are replaced with VVGFGSSPTS (SEQ ID NO: 13) or VVVVELSSPS (SEQ ID NO: 14), or any combination thereof: positions 72-78 of the polypeptide according to SEQ ID NO: 6 are replaced with HCHDEE (SEQ ID NO: 15) or YHVDRS (SEQ ID NO: 12), wherein positions 349-358 according to SEQ ID NO: 6 are replaced with VEVSLTSAQIA (SEQ ID NO: 16) or VVVVELSSPS (SEQ ID NO: 14), or any combination thereof: positions 73-78 of the polypeptide according to SEQ ID NO: 7 are replaced with HCHDEE (SEQ ID NO: 15) or TYHVDRS (SEQ ID NO: 11), wherein positions 349-358 according to SEQ ID NO: 7 are replaced with VEVSLTSAQIA (SEQ ID NO: 16) or VVGFGSSPTS (SEQ ID NO: 13), or any combination thereof.

In embodiments, the polypeptide comprises one or more post-translational modifications. For example, the post-translational modification is selected from the group consisting of PEGylation, sialylation, glycosylation, acetylation, acylation, lipid modification, palmitoylation, palmitate addition, phosphorylation, Fc-Ig fusion, and glycolipid modification.

In embodiments, the polypeptide is degylcosylated.

In embodiments, the polypeptide comprises at least one insertion, deletion, or mutation.

Aspects of the invention are also drawn towards a chimeric polypeptide or fragment thereof, wherein the chimeric polypeptide comprises an amino acid sequence at least 80% identical to MHLFAEATTKFTLELYRQLRESDNNIFYSPISMMTALAMLQLGAKGNTEKQIEKVLQ FNETTKKTTEKSAHCHDEENVHEQFQKLMTQLNKSNDAYDLKAANSIYGAKGFPFV QTFLEDIKEYYQANVESLDFEHAAEESEKKINSWVESQTNGKIKDLFPNGSLNRSTIM VLVNAVYFKGQWNHKFDEKHTTEEKFWLNKNTSKPVQMMKQNIEFNFMFLEDVQ AKIVEIPYKGKELSMIVLLPVEINGLKQLEEQLTADKLLEWTRAENMHMTELYLSLP RFKVDEKYDLPIPLEHMGMVDAFDPQKADFSGMSSTQGLVVSKVLHKSFVEVNEEG TEAAAATGVEVSLTSAQIAEDFCCDHPFLFFIIHRKTNSILFFGRISSP (SEQ ID NO: 18), wherein Serpinb3a chimeric positions 71-76 are replaced with TYHVDRS (SEQ ID NO: 11) or YHVDRS (SEQ ID NO: 12), wherein positions 346-356 are replaced with VVGFGSSPTS (SEQ ID NO: 13) or VVVVELSSPS (SEQ ID NO: 14), or any combination thereof; MNSLSEANTKFMFDLFQQFRKSKENNIFYSPISITSALGMVLLGAKDNTAQQIKKVLH FDQVTENTTGKAATYHVDRSGNVHHQFQKLLTEFNKSTDAYELKIANKLFGEKTYL FLQEYLDAIKKFYQTSVESVDFANAPEESRKKINSWVESQTNEKIKNLIPEGNIGSNTT LVLVNAIYFKGQWEKKENKEDTKEEKFWPNKNTYKSIQMMRQYTSFHFASLEDVQ AKVLEIPYKGKDLSMIVLLPNEIDGLQKLEEKLTAEKLMEWTSLQNMRETRVDLHLP RFKVEESYDLKDTLRTMGMVDIFNGDADLSGMTGSRGLVLSGVLHKAFVEVTEEGA EAAAATAVVGFGSSPTSTNEEFHCNHPFLFFIRQNKTNSILFYGRFSSP (SEQ ID NO: 19), wherein Serpinb3 chimeric positions 72-78 are replaced with HCHDEE (SEQ ID NO: 15) or YHVDRS (SEQ ID NO: 12), wherein positions 349-358 are replaced with VEVSLTSAQIA (SEQ ID NO: 16) or VVVVELSSPS (SEQ ID NO: 14), or any combination thereof: or MNSLSEANTKFMFDLFQQFRKSKENNIFYSPISITSALGMVLLGAKDNTAQQISKVLH FDQVTENTTEKAATYHVDRSGNVHHQFQKLLTEFNKSTDAYELKIANKLFGEKTYQ FLQEYLDAIKKFYQTSVESTDFANAPEESRKKINSWVESQTNEKIKNLFPDGTIGNDT TLVLVNAIYFKGQWENKFKKENTKEEKFWPNKNTYKSVQMMRQYNSFNFALLEDV QAKVLEIPYKGKDLSMIVLLPNEIDGLQKLEEKLTAEKLMEWTSLQNMRETCVDLHL PRFKMEESYDLKDTLRTMGMVNIFNGDADLSGMTWSHGLSVSKVLHKAFVEVTEE GVEAAAATAVVVVELSSPSTNEEFCCNHPFLFFIRQNKTNSILFYGRFSSP (SEQ ID NO: 20), wherein SerpinB4 chimeric positions 73-78 of the polypeptide are replaced with HCHDEE (SEQ ID NO: 15) or TYHVDRS (SEQ ID NO: 11), wherein positions 349-358 are replaced with VEVSLTSAQIA (SEQ ID NO: 16) or VVGFGSSPTS (SEQ ID NO: 13), or any combination thereof.

In embodiments, the polypeptide comprises one or more post-translational modifications. For example, the post-translational modification is selected from the group consisting of PEGylation, sialylation, glycosylation, acetylation, acylation, lipid modification, palmitoylation, palmitate addition, phosphorylation, Fc-Ig fusion, and glycolipid modification.

In embodiments, the polypeptide is degylcosylated.

In embodiments, the polypeptide comprises at least one insertion, deletion, or mutation.

Still further, aspects of the invention are drawn towards a nucleic acid encoding the polypeptide as described herein.

Also, aspects of the invention are drawn towards a vector comprising a nucleic acid as described herein.

Aspects of the invention are further drawn to a cell comprising a vector as described herein. For example, the cell is a plant cell, an animal cell, or an insect cell.

Aspects of the invention are drawn towards a wound healing formulation, wherein the formulation comprises a therapeutically effective amount of a polypeptide as described herein, or a combination thereof, and a pharmaceutically acceptable carrier, excipient or diluent. For example, the excipient comprises a hydrophilic polymer, saline solution, a sustained-release vehicle, dressing, viscosity increasing agent, ointment base, antimicrobial preservative, temperature sensing probe, pH sensing probe, emulsifying agent, solvent, or any combination thereof. he hydrophilic polymer comprises a hydrogel. For example, the hydrogel comprises chitosan, collagen, silk fibroin, carboxylated silk fibroin, silk sericin, glycerine, aloe vera, methyl paraben, hydrogenated castor oil, hyaluronic acid, polypeptides, pHEMA, pHPMA, or any combination thereof.

In embodiments, the formulation comprises a topical formulation.

In embodiments, the formulation comprises one or more additional active ingredients. For example, the one or more additional active ingredients comprises an antibiotic, a pain reliever, an anti-inflammatory, an anti-scarring agent, a moisturizer, a steroid, an immune modulator, or a growth factor.

In embodiments, the composition is formulated as an ointment, cream, lotion, suspension, aqueous solution, dispersion, salve, gel, spray, film, or paste.

Further, aspects of the invention are drawn towards a wound dressing comprising a therapeutically effective amount of a polypeptide as described herein or a wound healing formulation as described herein.

In embodiments, the polypeptide or wound healing formulation is added to, coated on, or embedded into the wound dressing.

Aspects of the invention are also drawn towards a method of treating a subject afflicted with a wound, the method comprising administering topically onto the wound a polypeptide as described herein or a wound healing formulation as described herein.

In embodiments, the wound is a dermal wound or ulcer, a chronic wound or ulcer, an infected wound or ulcer, a burn wound or ulcer, a diabetic wound or ulcer, a skin wound or ulcer, or a cutaneous wound or ulcer.

Also, aspects of the invention are drawn towards a kit comprising a polypeptide as described herein or a wound healing formulation as described herein.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows a structure of SerpinB3 RSCB 2ZV6. The structure of SerpinB3 (human) rendered from a crystal structure RSCB 2ZV6, indicating location of reactive center loop (yellow) and A-beta sheet (blue). Structure of SerpinB3/B4 showing the conserved serpin superfamily architecture of a reactive center loop (RCL:yellow) and core A-beta sheet (cyan). RSCB structure 2ZV6.

FIG. 2 shows Serpinb3a mRNA expression after wounding in mouse skin (dermal injury) or mouse tongue (mucosal injury) from NCBI GEO GSE23006 (Chen et al., BMC Genomics 2010). Serpinb3a mRNA expression is increased in mouse skin after dermal injury, but not in mouse tongue after mucosal injury.

FIG. 3 shows Serpinb3a mRNA expression in acute and diabetic wound migrating keratinocytes from NCBI GEO GSE141956. Serpinb3a mRNA expression is lower in diabetic wound migrating keratinocytes versus acute wound migrating keratinocytes.

FIG. 4 shows Serpinb3a protein expression in acute wounds in Balb/c mice. Serpinb3a protein expression in acute wounds (Balb/c mice) increases by 6 hrs and peaks at 2 days post-injury before coming down to nearly undetectable levels by day 7. This timeline “leads” events associated with EMT in dermal wound healing.

FIG. 5 shows a graph of % keratinocytes by days post-injury and the correlation of Serpinb3a dynamics with histologic markers of EMT. Quantification of E-cadherin loss and nuclear Slug activation in wound tongue keratinocytes compared with quantified protein levels of Serpinb3a (N=3 each group).

FIG. 6 shows consensus alignment of mouse Serpinb3a (SEQ ID NO: 5) with human SerpinB3 (SEQ ID NO: 6) and human SerpinB4 (isoform 1) (SEQ ID NO: 7). Consensus alignment (SEQ ID NO: 4) of mouse Serpinb3a with human SerpinB3 and human SerpinB4 (isoform 1) from T-Coffee server indicating amino acid homology between all three proteins, with a variable region at the reactive center loop P1/P1′ scissile bond.

FIG. 7 shows recombinant production of 6×His-Serpinb3a in E. coli BL21 (DE3). Recombinant production of 6×His-Serpinb3a in E. coli BL21 (DE3) supported by Coomassie blue staining and western blot for anti-6×His (“6×His” disclosed as SEQ ID NO: 17). Expression was performed at 37 C for 3 hours after IPTG induction. Both monomeric ˜45 kD and dimer ˜90 kD bands are observed by western blot.

FIG. 8 shows a schematic of a healthy wound and a diabetic wound. Without wishing to be bound by theory, SerpinB3/B4 (mouse Serpinb3a) mediates epidermal EMT and re-epithelialization, which is impaired in diabetic wounds and can be therapeutically supplemented.

FIG. 9 shows Serpinb3a is induced upon skin injury. (Panel A) Time course analysis of Serpinb3a gene expression from mice with 1-mm dermal (black circles) or mucosal (pink squares) wounds at described times normalized to unwounded tissue. N=3/group, multiple T-test, **** p<0.0001. Data from NCBI GEO Dataset GSE23006 [31]. (Panel B) Immunoblot of Serpinb3a protein levels in intact skin (N=2) and from mice with 5-mm dermal wounds at 2- and 4-days post-wounding (N=3/ea). The membrane was post-stained with Coomassie Blue for total protein control.

FIG. 10 shows migrating keratinocytes from db/db mice have lower expression of Serpinb3a. Relative Serpinb3a gene expression in laser-captured migrating keratinocytes at 3 days post wounding in C57BL6/J wildtype and db/db mice. N=3/ea, T-test, *p<0.05.

FIG. 11 shows a graph and in vitro images of non-limiting, exemplary results of treating scratch wounds with a control, mEGF, or Serpinb3a. Serpinb3a accelerates wound closure in vitro. Scratch wounds in a HaCaT keratinocyte monolayer were untreated or treated with mEGF or Serpinb3a. Significant closure was observed at 24 hr vs. controls with both treatments, indicating Serpinb3a is a potent pro-healing agent. N=2 with duplicates.

FIG. 12 shows graphs of non-limiting, exemplary results of acute and diabetic wound closure in vivo. Serpinb3a accelerates acute and diabetic wound closure in vivo with improved barrier function recovery in diabetic mice. (Panel A) Wound planimetry of full-thickness dermal wounds on C57BL6/J mice (black circles) and db/db diabetic mice (pink squares) treated with saline (closed symbols) or 500 ng/g bodyweight recombinant Serpinb3a (open symbols) during the reepithelialization phase of healing (days 0-6). (Panel B) Transepidermal water loss (TEWL) measurements at terminal follow-up (day 14 for C57BL6/J and day 28 for db/db mice). N=2-6 per group.

FIG. 13 shows an illustration of, without wishing to be bound by theory, a mechanism of wound healing.

FIG. 14 shows non-limiting, exemplary data of Serpinb3a expression.

FIG. 15 shows non-limiting, exemplary human keratinocyte expression data.

FIG. 16 shows non-limiting, exemplary cell proliferation and morphology data.

FIG. 17 shows non-limiting, exemplary production and validation data of recombinant Serpinb3a. Panel A shows a non-limiting, exemplary SDS-PAGE and Western blot. The SDS-PAGE/Coomassie shows elution yield from 500 mL culture: 8.7 mg/mL >85% pure. Panel B shows a non-limiting, exemplary graph of A/A₀ (405 nm) vs. time for Cathepsin G and a control (adapted from Yaron et al. Methods Mol. Bio. 2018). Panel C shows a non-limiting, exemplary graph of A/A₀ (405 nm) vs. time. Panel D shows a non-limiting, exemplary graph of Fractional Velocity vs. log ([I]₀/[E]₀). FIG. 17 discloses “6×His” as SEQ ID NO: 17.

FIG. 18 shows a Western blot. rSerpinb3a reversibly dimerizes in solution.

FIG. 19 shows non-limiting, exemplary illustrations and data of protein polishing of Serpinb3a. Panel A shows a non-limiting, exemplary illustration of process steps. Panel B shows a picture of LAL Gel Clot Assay Results. Panel C shows a non-limiting, exemplary graph of A/A0 (405 nm) vs. time. 1:1 inhibitory stoichiometry is maintained after endotoxin removal and buffer exchange. Panel D shows a non-limiting, exemplary bar graph of QUANTI-Luc and QUANTI-Blue. Serpinb3a is not intrinsically inflammatory. FIG. 19 discloses “6×His” as SEQ ID NO: 17.

FIG. 20 shows non-limiting, exemplary imaging stills of in an in vitro scratch assay using human HaCaT keratinocytes. Live cell imaging was performed to visualize the dynamic migrating front of the epithelial sheet. Compared to control, exogenous Serpinb3a treatment (100 ng/mL) promoted an enhanced migratory behavior of epithelial cells into the scratch wound over a period of 24 hours, similar to the positive control mEGF (1 ng/ml). The yellow line indicates the beginning edge of the scratch wound.

FIG. 21 shows non-limiting, exemplary images of a control, 1 ng/mL mEGF, and 0.1 μg/mL Serpinb3a. Panel A shows staining with Phalloidin CF488. Panel B shows staining with H33342 and Phalloidin CF488.

FIG. 22 shows non-limiting, exemplary graphs of data. Panel A shows a graph of wound area (% initial) vs. days post-injury. Panel B shows a bar graph of TEWL (g/m² h) vs treatment.

FIG. 23 shows non-limiting, exemplary graphs and images of an intact sample, a saline treated sample, and Serpinb3a treated sample.

### DETAILED DESCRIPTION OF THE INVENTION

Large surface wounds, including lacerations and burns, are common injuries that can be complex. In some cases, comorbidities such as diabetes and advanced age cause skin lesions to turn into non-healing chronic wounds, reducing function and increasing risk of infection and bleeding. Chronic non-healing wounds can be life threatening and are a major threat to public health and a large cost to the economy (Sen et al., Wound Repair Regen. 17 (2009) 763-71, Nussbaum et al., Value Health. 21 (2018) 27-32). According to the NIH ARRA Impact Report, over 6 million cases of chronic wounds occur annually in the United States with a collective cost of more than $20 billion per year. Severe burn injuries cause about 40,000 hospitalizations and nearly 4,000 deaths each year. These numbers do not include scar revisions, which amount to over 170,000 procedures annually in the USA (Lim et al., Plast. Reconstr. Surg. 133 (2014) 398-405).

The wound healing process is frequently divided into three steps: hemostasis and inflammation, new tissue generation and remodeling (Eming et al., Sci. Transl. Med. 6 (2014)). The immune system plays a central role in each step. Wound healing in adults can begin with bleeding and clot formation (haemostasis) followed by a rapid-onset of inflammation. Immune response cells, including neutrophils (Wilgus et al., Adv. Wound Care. (2013), Soehnlein et al., Nat. Rev. Immunol. (2017)) and macrophages (Lucas et al., J. Immunol. 184 (2010) 3964-3977, Hesketh et al., Int. J. Mol. Sci. (2017), Wynn and Vannella, Immunity. 44 (2016) 450-462, Mantovani et al., J. Pathol. (2013), Brancato and AlbinaAm. J. Pathol. 178 (2011) 19-25) are known to be crucial in initiating the early stage of wound healing.

In embodiments acute inflammation can be critical to healthy wound healing, with innate immunity driving early responses to injury and with precisely regulated stages at both the cellular and molecular levels, while sustained and excessive inflammation can exacerbate damage and result in chronic wounds (Eming et al., J. Invest. Dermatol. (2007), Landen et al., Cell. Mol. Life Sci. (2016)).

In embodiments intrinsic reparative responses can be critical to healthy wound healing. As used herein, the term “intrinsic reparative responses” can refer to processes a body, organ, tissue, and/or cell performs in response to a disruption, trauma, and/or damage which results in or promotes restoration or healing of the body, organ, tissue, or cell. For example, the intrinsic reparative response can comprise epithelial-to-mesenchymal transition (EMT) and proliferation pathways. For example, EMT can refer to a process in which epithelial cells lose their cell polarity and cell adhesion ability and acquire invasiveness and migration to become mesenchymal-like cells. In embodiments, proliferation pathways can refer to cell proliferation pathways. For example, a cell proliferation pathway can refer to any pathway in a process by which a cell divides.

In embodiments, Serpinb3a can act on intrinsic reparative responses. For example, the intrinsic reparative responses can comprise epithelial-to-mesenchymal transition (EMT). For example, the intrinsic reparative responses can comprise proliferation pathways.

Modulating the immune system or intrinsic reparative responses through biomaterials and drug delivery systems can alter wound healing, increasing regeneration and reducing fibrosis (Zhao etal., Int. J. Mol. Sci. (2016); Julier et al., Acta Biomater. (2017); Stejskalova and Almquist, Biomater. Sci. 5 (2017) 1421-1434). The three major factors that fundamentally alter wound healing and management are infection, wound closure and fibrosis (scarring). Wound healing in chronic or infected wounds is a major health problem causing morbidity and increasing mortality in diabetic or burn patients.

As described herein, recombinant serine protease inhibitor polypeptides and fragments thereof are engineered and adapted as a new protein biologic for promoting wound healing. Aspects of the invention are drawn to formulations, such as topical formulations, for promoting wound healing, wherein the formulation comprises a therapeutically effective amount of a recombinant serine protease inhibitor polypeptide. Aspects of the invention are also drawn to a wound dressing or bandage comprising a therapeutically effective amount of a recombinant serine protease inhibitor polypeptide. For example, the wound dressing or bandage comprises the formulation described herein. Still further, aspects of the invention are drawn to methods of treating a wound in a subject, such as a chronic wound, a dermal wound, or an infected wound.

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A Meyers (ed.), Molecular Biology and Biotechnology: a Comprehensive Desk Reference, published by VCH Publishers, Inc., 1995 (ISBN 9780471185710); and other similar references.

Suitable methods and materials for the practice or testing embodiments of the invention are described herein. Such methods and materials are illustrative only and are not intended to be limiting. Although methods and materials similar or equivalent to those described herein can be used in the practice or testing of embodiments of the invention, suitable methods and materials are described herein. All publications, patent applications, patents, and other references mentioned herein are incorporated by reference in their entirety.

Unless otherwise defined, all technical terms used herein have the same meaning as commonly understood. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999. In addition, the materials, methods, and examples are illustrative only and not intended to be limiting.

### Recombinant Serine Protease Inhibitor (Serpin) Polypeptides

Aspects of the invention are drawn towards recombinant serine protease inhibitor (serpin) superfamily polypeptides. A “peptide”, “polypeptide”, and/or “protein”, which can be used interchangeably, can refer to any compound composed of amino acids or amino acid analogs that are chemically bound together. For example, amino acids can be chemically bound together via amide linkages (CONH), or can be bound together by other chemical bonds (such as amine linkages). Peptides can include oligomers of amino acids, amino acid analogs, or small and large peptides, including polypeptides or proteins.

The term “recombinant polypeptide” can refer to a polypeptide that is produced by recombinant techniques, for example wherein DNA or RNA encoding the expressed protein is inserted into a suitable expression vector that is in turn used to transform a host cell to produce the polypeptide.

Embodiments can comprise a recombinant polypeptide or fragment thereof corresponding to Serpinb3a (mouse-derived). Such an embodiment can retain the function of both human SerpinB3 and human SerpinB4. For example, such embodiments can comprise a polypeptide according to the amino acid:

(SEQ ID NO: 1)
MHLFAEATTKFTLELYROLRESDNNIFYSPISMMTALAMLQLGAKGNTEK
QIEKVLQFNETTKKTTEKSAHCHDEENVHEQFQKLMTQLNKSNDAYDLKA
ANSIYGAKGFPFVQTFLEDIKEYYQANVESLDFEHAAEESEKKINSWVES
QTNGKIKDLFPNGSLNRSTIMVLVNAVYFKGQWNHKFDEKHTTEEKFWLN
KNTSKPVQMMKQNIEFNFMFLEDVQAKIVEIPYKGKELSMIVLLPVEING
LKQLEEQLTADKLLEWTRAENMHMTELYLSLPRFKVDEKYDLPIPLEHMG
MVDAFDPQKADFSGMSSTQGLVVSKVLHKSFVEVNEEGTEAAAATGVEVS
LTSAQIAEDFCCDHPFLFFIIHRKINSILFFGRISSP
(NP_033152.3)

Embodiments can further comprise a recombinant polypeptide or fragment thereof corresponding to human SerpinB3 and/or SerpinB4:

(SEQ ID NO: 2)
MNSLSEANTKFMFDLFQQFRKSKENNIFYSPISITSALGMVLLGAKDNTA
QQIKKVLHFDQVTENTTGKAATYHVDRSGNVHHQFQKLLTEFNKSTDAYE
LKIANKLFGEKTYLFLQEYLDAIKKFYQTSVESVDFANAPEESRKKINSW
VESQTNEKIKNLIPEGNIGSNTTLVLVNAIYFKGQWEKKFNKEDTKEEKF
WPNKNTYKSIQMMRQYTSFHFASLEDVQAKVLEIPYKGKDLSMIVLLPNE
IDGLQKLEEKLTAEKLMEWTSLQNMRETRVDLHLPRFKVEESYDLKDTLR
TMGMVDIFNGDADLSGMTGSRGLVLSGVLHKAFVEVTEEGAEAAAATAVV
GFGSSPTSTNEEFHCNHPFLFFIRQNKTNSILFYGRFSSP.
(NP_008850.1)
(SEQ ID NO: 3)
MNSLSEANTKFMFDLFQQFRKSKENNIFYSPISITSALGMVLLGAKDNTA
QQISKVLHFDQVTENTTEKAATYHVDRSGNVHHQFQKLLTEFNKSTDAYE
LKIANKLFGEKTYQFLQEYLDAIKKFYQTSVESTDFANAPEESRKKINSW
VESQTNEKIKNLFPDGTIGNDTTLVLVNAIYFKGQWENKFKKENTKEEKF
WPNKNTYKSVQMMRQYNSFNFALLEDVQAKVLEIPYKGKDLSMIVLLPNE
IDGLQKLEEKLTAEKLMEWTSLQNMRETCVDLHLPRFKMEESYDLKDTLR
TMGMVNIFNGDADLSGMTWSHGLSVSKVLHKAFVEVTEEGVEAAAATAVV
VVELSSPSTNEEFCCNHPFLFFIRQNKTNSILFYGRFSSP.
(NP_002965.1)

In embodiments, the sequences described herein can comprise a glycine linker and C-terminal 6×-Histag (SEQ ID NO: 17). Such modifications can be helpful for peptide purification, for example.

Amino acid sequence functional variants of the polypeptide can be prepared by mutations in the DNA which encodes the polypeptide. Such variants or functional variants include, for example, deletions from, or insertions or substitutions of, residues within the amino acid sequence. Any combination of deletion, insertion, and substitution can also be made to arrive at the final recombinant polypeptide, provided that the final recombinant polypeptide possesses the target activity.

For example, embodiments can comprise consensus mutants comprising substitutions from the human SerpinB3 and/or SerpinB4. The phrase “consensus mutant” can refer to a mutant version of SerpinB3 and/or SerpinB4 in which individual amino acid residues are mutated to the one which occurs most frequently at that site in the compared human and mouse sequences.

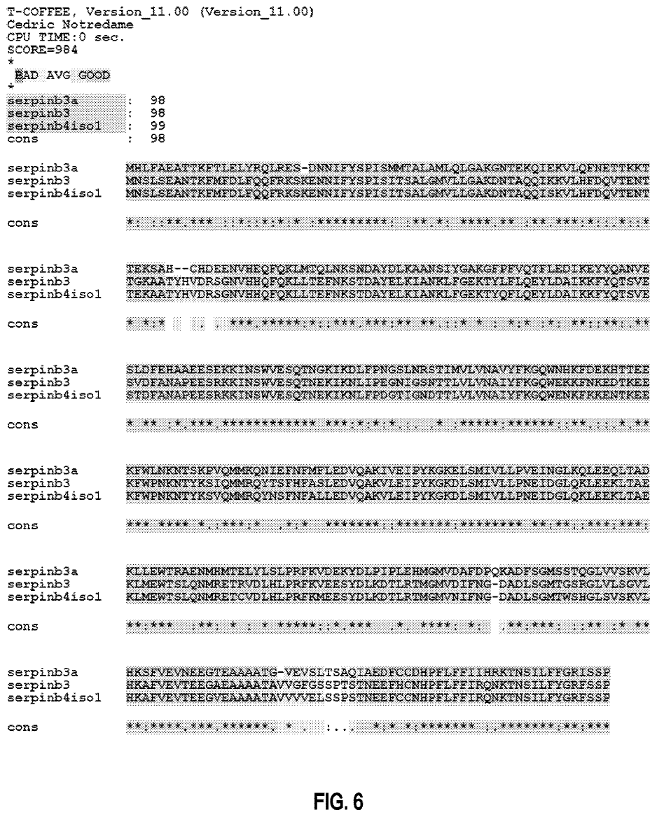

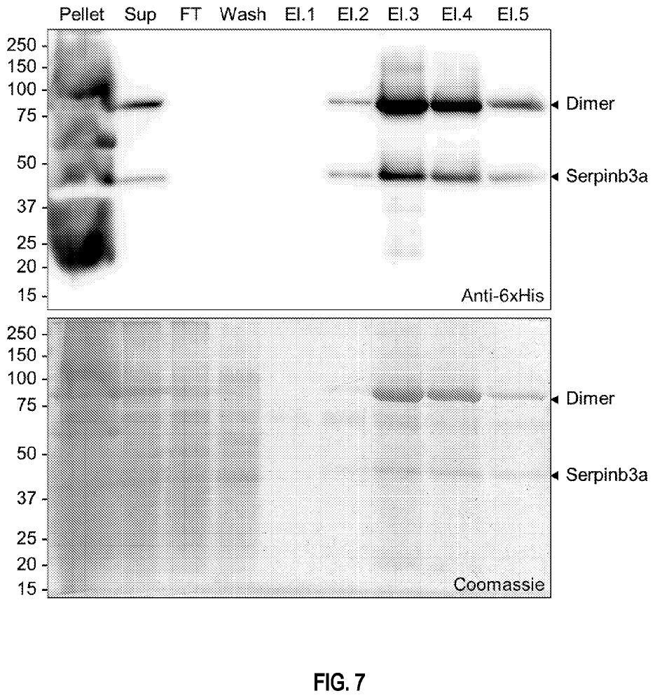

As another example, embodiments can comprise N-terminal variable region chimeras or reactive center loop truncation mutants of Serpinb3a, SerpinB3, or SerpinB4. As used herein, the terms “reactive center loop truncation mutant” and “reactive center loop chimeras” can be used interchangeably. Referring to FIG. 6, for example, the N-terminal variable region chimeras comprise Serpinb3a, SerbinB3, or SerpinB4 chimeric positions at about position 70 to about position 80 of the polypeptide according to SEQ ID NO: 4 (consensus sequence, represented by “cons”) replaced with TYHVDRS (SEQ ID NO: 11), HCHDEE (SEQ ID NO: 15), YHVDRS (SEQ ID NO: 12), or any combination thereof. For example, the N-terminal variable region chimeras comprise Serpinb3a chimeric positions at 71-76 of the polypeptide according to serpinb3a (such as the amino acid sequence according to FIG. 6, SEQ ID NO: 5) replaced with TYHVDRS (SEQ ID NO: 11), YHVDRS (SEQ ID NO: 12), or any combination thereof. For example, the N-terminal variable region chimeras comprise SerpinB3 chimeric positions at 72-78 of the polypeptide according to serpinb3 (such as the amino acid sequence according to FIG. 6, SEQ ID NO: 6) replaced with HCHDEE (SEQ ID NO: 15), YHVDRS (SEQ ID NO: 12), or any combination thereof. In embodiments, the N-terminal variable region chimeras comprise SerpinB4 positions 73-78 of the polypeptide according to serpinB4 (such as the amino acid sequence according to FIG. 6, SEQ ID NO: 7) are replaced with HCHDEE (SEQ ID NO: 15), TYHVDRS (SEQ ID NO: 11), or any combination thereof. In embodiments, the reactive center loop chimeras comprise Serpinb3a, SerpinB3, or SerpinB4 chimeric positions at about position 340 to about position 360 of the polypeptide according to SEQ ID NO: 4 (consensus sequence, represented by “cons”) replaced with VVGFGSSPTS (SEQ ID NO: 13), VVVVELSSPS (SEQ ID NO: 14), VEVSLTSAQIA (SEQ ID NO: 16), or any combination thereof. For example, the reactive center loop chimeras comprise Serpinb3a chimeric positions 346-356 of the polypeptide according serpinb3a (such as the amino acid sequence according to FIG. 6, SEQ ID NO: 5) replaced with VVGFGSSPTS (SEQ ID NO: 13), VVVVELSSPS (SEQ ID NO: 14), or any combination thereof. For example, the reactive center loop chimeras comprise SerpinB3 chimeric positions 349-358 of the polypeptide according to serpinb3 (such as the amino acid sequence according to FIG. 6, SEQ ID NO: 6) replaced with VEVSLTSAQIA (SEQ ID NO: 16), VVVVELSSPS (SEQ ID NO: 14), or any combination thereof. For example, the reactive center loop chimeras comprise SerpinB4 chimeric positions 349-358 of the polypeptide according serpinB4 (such as the amino acid sequence according to FIG. 6, SEQ ID NO: 7) replaced with VEVSLTSAQIA (SEQ ID NO: 16), VVGFGSSPTS (SEQ ID NO: 13), or any combination thereof. As yet another example, embodiments can comprise consensus mutants containing relevant substitutions into Serpinb3a from human SerpinB3 or SerpinB4 in addition to reactive center loop truncation.

Embodiments as described herein can comprise a polypeptide fragment. The term “fragment” of a polypeptide can refer to a shorter portion of a full-length polypeptide or protein ranging in size from four amino acid residues to the entire amino acid sequence minus one amino acid residue. For example, biologically active fragments can include polypeptides of about 4, about 10, about 15, about 20, about 25, about 30, about 35, about 40, about 45, about 50, or greater than 50 amino acids. In certain embodiments of the disclosure, a fragment can refer to the entire amino acid sequence of a domain of a polypeptide or protein (e.g., a substrate binding domain or a catalytic domain).

For example, the polypeptide fragment can comprise the sequence according to:

(SEQ ID NO: 8)
GTEAAAATGVEVSLTSAQIA
(Serpinb3a), NP_033152.3;
(SEQ ID NO: 9)
GAEAAAATAVVGFGSSPTST
(SerpinB3), NP_008850.1;
(SEQ ID NO: 10)
GVEAAAATAVVVVELSSPST
(SerpinB4), NP_002965.1.

Embodiments of the invention also comprise a chimeric polypeptide or fragment thereof. As used herein, the term “chimeric” can refer to a polypeptide that contains portions from at least two different polypeptides or from two non-contiguous portions of a single polypeptide. For example, a chimeric polypeptide of the invention can comprise:
an amino acid sequence at least 80% identical to SEQ ID NO: 1 (Serpinb3a mouse derived) (NP_033152.3), wherein positions 71-76 are replaced with TYHVDRS (SEQ ID NO: 11) or YHVDRS (SEQ ID NO: 12), wherein positions 346-356 are replaced with VVGFGSSPTS (SEQ ID NO: 13) or VVVVELSSPS (SEQ ID NO: 14), or any combination thereof;
an amino acid sequence at least 80% identical to SEQ ID NO: 2 (human SerpinB3) (NP_008850.1), wherein chimeric positions 72-78 are replaced with HCHDEE (SEQ ID NO: 15) or YHVDRS (SEQ ID NO: 12), wherein positions 349-358 are replaced with VEVSLTSAQIA (SEQ ID NO: 16) or VVVVELSSPS (SEQ ID NO: 14), or any combination thereof; or
an amino acid sequence at least 80% identical to SEQ ID NO: 3 (human SerpinB4) (NP_002965.1), wherein chimeric positions 73-78 are replaced with HCHDEE (SEQ ID NO: 15) or TYHVDRS (SEQ ID NO: 11), wherein positions 349-358 are replaced with VEVSLTSAQIA (SEQ ID NO: 16) or VVGFGSSPTS (SEQ ID NO: 13), or any combination thereof.

Minor variations in the amino acid sequences of polypeptides can be encompassed by the inventive concept(s) disclosed and claimed herein, providing that the variations in the amino acid sequence maintain at least 80% sequence identity, such as at least 80%, 81%, 82%, 83%, 84%, 85%, 86%, 87%, 88%, 89%, 90%, 91%, 92%, 93%, 94%, 95%, 96%, 97%, 98% and 99% sequence identity. The polypeptides can be modified specifically to alter a feature of the polypeptide unrelated to its physiological activity. For example, certain amino acids can be changed and/or deleted without affecting the physiological activity of the polypeptide in this study (i.e., its ability to induce a tumor-specific immune response).

Conservative amino acid replacements are those that take place within a family of amino acids that are related in their side chains. Genetically encoded amino acids can be divided into families: (1) acidic=aspartate, glutamate: (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Other families include serine and threonine are aliphatic-hydroxy family; asparagine and glutamine are an amide-containing family: alanine, valine, leucine and isoleucine are an aliphatic family; and phenylalanine, tryptophan, and tyrosine are an aromatic family. For example, without wishing to be bound by theory, an isolated replacement of a leucine with an isoleucine or valine, an aspartate with a glutamate, a threonine with a serine, or a similar replacement of an amino acid with a structurally related amino acid can not have a major effect on the binding or properties of the resulting molecule, especially if the replacement does not involve an amino acid within a framework site. Whether an amino acid change results in a functional peptide can readily be determined by assaying the specific activity of the peptide derivative. Fragments or analogs of proteins/peptides can be readily prepared by those of ordinary skill in the art. Exemplary amino- and carboxy-termini of fragments or analogs occur near boundaries of functional domains. In one example, one amino acid residue (e.g., valine) of a synthetic peptide can be conservatively replaced (e.g., by leucine). In other examples, two amino acid residues of a synthetic peptide can be conservatively replaced by other suitable amino acid residues, for example, valine (V) and arginine (R) are replaced by the pair of amino acids that includes, but is not limited to, methionine (M) and lysine (K), lysine (K) and proline (P), tryptophan (W) and isoleucine (I), isoleucine (I) and proline (P), asparagine (N) and valine (V), and glutamine (G) and lysine (K).

Recombinant polypeptides as described herein can comprise synthetic polypeptides. The term “synthetic polypeptide” can refer to a polypeptide which does not comprise an entire naturally occurring protein molecule. The polypeptide is “synthetic” in that it can be produced by human intervention using such techniques as chemical synthesis, recombinant genetic techniques, or fragmentation of whole antigen or the like.

Further, recombinant polypeptides can also comprise analogs (non-peptide organic molecules), derivatives (chemically functionalized peptide molecules obtained starting with the disclosed peptide sequences), mutants, and variants (homologs) of the peptides disclosed herein, which can be utilized in the formulations and methods described herein. Each peptide of this disclosure is comprised of a sequence of amino acids, which can be L- and/or D-amino acids, naturally occurring and otherwise.

The recombinant peptides as described herein can be modified by a variety of chemical techniques to produce derivatives having essentially the same activity as the unmodified peptides, and optionally having other desirable properties. For example, carboxylic acid groups of the peptide, whether carboxyl-terminal or side chain, can be provided in the form of a salt of a pharmaceutically-acceptable cation or esterified to form a Cl-Cl 6 ester, or converted to an amide of formula NR1R2 wherein RI and R2 are each independently H or Cl-Cl 6 alkyl, or combined to form a heterocyclic ring, such as a 5- or 6-membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to Cl-Cl 6 alkyl or dialkyl amino or further converted to an amide.

Hydroxyl groups of the peptide side chains can be converted to Cl-Cl 6 alkoxy or to a Cl-Cl 6 ester using well-recognized techniques. Phenyl and phenolic rings of the peptide side chains can be substituted with one or more halogen atoms, such as fluorine, chlorine, bromine or iodine, or with Cl-Cl 6 alkyl, Cl-Cl 6 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides to select and provide conformational constraints to the structure that result in enhanced stability. While the peptides of the disclosure can be linear or cyclic, cyclic peptides can have an advantage over linear peptides in that their cyclic structure is more rigid and hence their biological activity can be higher than that of the corresponding linear peptide. Any method for cyclizing peptides can be applied to the serpin-derived peptides or fragments described herein.

In embodiments, the recombinant polypeptide or fragment thereof comprises one or more post-translational modifications or modifications thereof. Examples of such post-translational modifications include, but are not limited to, glycosylation, deglycosylation, sialylation, acetylation, acylation, lipid modification, palmitoylation, palmitate addition, phosphorylation, glycolipid modification, PEGylation, methylation, Fc-Ig fusion, and the like. For example, the Fc-Ig fusion can comprise N- or C-terminal fusions to the Fc constant regions of the human IgG1 protein. In embodiments, Fc-Ig fusion can enhance solubility, half-life, and function. In embodiments, the cell line that produces the recombinant serine protease inhibitor polypeptide and/or the culture conditions can change the post-translational modification profile and activity of the recombinant serine protease inhibitor polypeptide or fragment thereof.

For example, the peptide modification can be PEGylation, or linking of the recombinant polypeptide to polyethylene glycol, so as to increase solubility and prolong circulatory time. Once linked to a peptide, the PEG subunit becomes tightly associated with two or three water molecules, which has the dual function of rendering the polypeptide more soluble in water and making its molecular structure larger. As the kidneys filter substances according to size, the addition of PEG's molecular weight can prevent the premature renal clearance undergone by small peptides. PEG's globular structure can also act as a shield to protect the polypeptide of the invention from proteolytic degradation, and can reduce the immunogenicity of foreign peptides by limiting their uptake through the dendritic cells. PEG itself is not immunogenic or toxic, and allows for lower doses and less-frequent administrations. In some instances, PEG can increase the circulating half-life of a peptide drug by more than 100 times. In addition to improving the pharmacokinetic and pharmacodynamic properties of peptide drugs once inside the body, PEGylation can also aid drug delivery because PEGylated peptides act as permeation enhancers for nasal drug delivery.

In embodiments, the PEG molecule can be monomethoxy PEG (mPEG), which has relatively simple chemistry due to its monofunctionality (CH3O—(CH2CH2O)n-CH2CH2-OH). In other embodiments, the PEG molecule can be HiPEG, or PEG attached to histidine sequences expressed on the N or C terminal of proteins. For example, 6 His-tags (SEQ ID NO: 17) can be used to create site-specific PEGylated conjugates, that is, PEGylation using a His-tagging approach. A protein is encoded with a polyhistidine tag (such as a 6 histidine tag (SEQ ID NO: 17)). Once incubated with a Ni-nitrilotriacetic acid (NTA)-PEG reagent, a complex is formed between the histidine residues and the nickel ion, thus PEGylating the protein. In other embodiments, the PEG molecule can be branched or forked PEG, such as PEG2, releasable PEGs (rPEGs), or heterbifunctional PEGs, details of which can be found in Roberts, M. J., M. D. Bentley, and J. M. Harris. “Chemistry for peptide and protein PEGylation.” Advanced drug delivery reviews 64 (2012): 116-127. One of ordinary skill in the art appreciates the routine methods practiced to pegylate amino acid residues of peptides of interest.

In embodiments, the peptide modification can be methylation. The methylation of proteins, for example, can help regulate cellular functions such as transcription, cell division, and cell differentiation. Methylation of the recombinant serine protease inhibitor polypeptide, for example, can extend the half-life of the peptides. Methylation of amino acid residues can be performed according to methods well understood by one of ordinary skill in the art (see. for example, US20090264620 and Mini Rev Med Chem. 2016; 16 (9): 683-90).

In embodiments, the peptide modification can be amidation or acetylation, such as at the C terminus or N terminus, respectively. Such modifications can also increase the metabolic stability of the peptides, as well as their ability to resist enzymatic degradation by aminopeptidases, exopeptidases, and synthetases. Amidation and acetylation of amino acid residues can be performed according to methods well understood by the skilled artisan. See. for example, Cottingham, Ian R., et al. “A method for the amidation of recombinant peptides expressed as intein fusion proteins in Escherichia coli.” Nature biotechnology 19.10 (2001): 974-977; Cerovsky, Václav, and Maria-Regina Kula. “Peptide amidase-catalyzed C-terminal peptide amidation in a mixture of organic solvents.” Peptides for the New Millennium (2002): 142-143; Mura, Manuela, et al. “The effect of amidation on the behaviour of antimicrobial peptides.” European Biophysics Journal 45.3 (2016): 195-207; Thomas, A., Towards a Functional Understanding of Protein N-Terminal Acetylation. PLOS Biol. 2011, 9 (5); and Wallace, R. J., Acetylation of peptides inhibits their degradation by rumen micro-organisms. British Journal of Nutrition. 1992, 68, 365-372.

In embodiments, the peptide modification can be acetylation, for example N-terminal acetylation (see, for example, U.S. Pat. No. 9,062,093). This modification makes the resulting peptide more stable towards enzymatic degradation resulting from exopeptidases.

Peptidomimetic and organomimetic embodiments are encompassed herein, whereby the three-dimensional arrangement of the chemical constituents of such peptido- and organomimetics mimic the three-dimensional arrangement of the peptide backbone and component amino acid side chains, resulting in such peptido- and organomimetics of a peptide having measurable recombinant serine protease inhibitor polypeptide activity. For computer modeling applications, a pharmacophore is an idealized three-dimensional definition of the structural requirements for biological activity. Peptido- and organomimetics can be designed to fit each pharmacophore with current computer modeling software.

In embodiments, the recombinant polypeptide or fragment thereof can be included in a fusion protein. For example, the fusion protein can include the recombinant polypeptide or fragment thereof and a second heterologous moiety, such as a myc protein, an enzyme or a carrier (such as a hepatitis carrier protein or bovine serum albumin) covalently linked to the recombinant serine protease inhibitor polypeptide or fragment thereof. A second heterologous moiety can be covalently or non-covalently linked to the recombinant polypeptide or fragment thereof. The recombinant polypeptide or fragment thereof can be included in a fusion protein and can also include heterologous sequences.

In embodiments, the recombinant polypeptide or fragment thereof can be conjugated to a macromolecule, non-limiting examples of which comprise carrier proteins such as keyhole limpet hemocyanin (KLH), tetanus toxoid (TT), or bovine serum albumin (BSA). Conjugation of the peptide to such molecules, for example, can increase the stability of the peptide, or can increase resistance to proteolytic cleavage. Conjugation methods as listed herein are well understood by the skilled artisan (Chapter 3 Peptide-carrier conjugation: Laboratory Techniques in Biochemistry and Molecular Biology: Volume 19, 1988, Pages 95-130).

“Conjugation” can refer to the linking of a peptide, directly or indirectly, to another molecule. For example, “direct conjugation” can refer to linking of the recombinant serine protease inhibitor polypeptide to an activated carbohydrate, another antigenic universal peptide, or a peptide linker, without introducing additional functional groups. As another example, “indirect conjugation” can refer to the addition of functional groups which are used to facilitate conjugation. For example, carbohydrate can be functionalized with amines which are subsequently reacted with bromoacetyl groups. The bromoacetylated carbohydrate is then reacted with thiolated protein. (Hermanson, GT, Bioconjugate Techniques, Academic Press, 2nd ed, 2008). The term “functionalization” can mean to chemically attach a group to add functionality, for example, to facilitate conjugation. Examples include functionalization of proteins with hydrazides or aminooxy groups and functionalization of carbohydrate with amino groups.

Embodiments of the invention can comprise two or more polypeptides that are linked to each other (i.e., crosslinked). “Crosslinking” can refer to joining moieties together, such as recombinant serine protease inhibitor polypeptides, by noncovalent or covalent bonds. For example, two or more polypeptides can be covalently linked by a linker. In embodiments, the linker can be a peptide linker. For example, the crosslinking comprises covalent crosslinking between polymers (i.e., polypeptides).

Embodiments can also comprise nucleic acids encoding one or more recombinant polypeptides or fragments thereof. These polynucleotides can include DNA, cDNA and RNA sequences which encode the peptide(s) of interest. Nucleic acid molecules encoding these peptides can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same peptide.

Nucleic acid sequences encoding one or more recombinant polypeptides can be prepared by any suitable method including, for example, cloning of appropriate sequences or by direct chemical synthesis by methods known to the skilled artisan. See, for example, the phosphotriester method of Narang et al., Meth. Enzymol. 68:90-99, 1979; the phosphodiester method of Brown et al, Meth. Enzymol. 68:109-151, 1979; the diethylphosphoramidite method of Beaucage et al, Tetra. Lett. 22:1859-1862, 1981 the solid phase phosphoramidite triester method described by Beaucage & Caruthers, Tetra. Letts. 22 (20): 1859-1862, 1981, a method using an automated synthesizer as described in, for example, Needham-VanDevanter et al, Nucl. Acids Res. 12:6159-6168, 1984; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template.

Exemplary nucleic acids including sequences encoding one or more recombinant polypeptides disclosed herein can be prepared by cloning techniques or chemical synthesis. Examples of appropriate cloning and sequencing techniques, and instructions sufficient to direct persons of skill through cloning are found in Sambrook et al, supra, Berger and Kimmel (eds.), supra, and Ausubel, supra. Product information from manufacturers of biological reagents and experimental equipment also provide useful information. Such manufacturers include the SIGMA Chemical Company (Saint Louis, MO), R&D Systems (Minneapolis, MN), Pharmacia Amersham (Piscataway, NJ), CLONTECH Laboratories, Inc. (Palo Alto, CA), Chem Genes Corp., Aldrich Chemical Company (Milwaukee, WI), Glen Research, Inc., GIBCO BRL Life Technologies, Inc. (Gaithersburg, MD), Fluka Chemica-Biochemika Analytika (Fluka Chemie AG, Buchs, Switzerland), Invitrogen (San Diego, CA), and Applied Biosystems (Foster City, CA), as well as many other commercial sources known to one of skill.

Once the nucleic acids encoding one or more recombinant polypeptides are isolated and cloned, the peptide can be expressed in a recombinantly engineered cell such as bacteria, plant, yeast, insect and mammalian cells using a suitable expression vector or expressed in a viral vector for therapeutic approaches, such as adeno-associated viral (AAV) vector expression. One or more DNA sequences encoding one or more peptides can be expressed in vitro by DNA transfer into a suitable host cell. The cell can be prokaryotic or eukaryotic. The term also includes any progeny of the subject host cell. In embodiments, the progeny are not identical to the parental cell since there can be mutations that occur during replication. Methods of stable transfer, meaning that the foreign DNA is continuously maintained in the host, are known in the art. In one example a vector is an adeno-associated virus (AAV) vector.

The terms “recombinant nucleic acid” or “recombinantly produced nucleic acid” can refer to nucleic acids such as DNA or RNA which has been isolated from its native or endogenous source, and which can be modified, for example, chemically or enzymatically, by adding, deleting or altering naturally-occurring flanking or internal nucleotides. Flanking nucleotides are those nucleotides which are upstream or downstream from the described sequence or sub-sequence of nucleotides, while internal nucleotides are those nucleotides which occur within the described sequence or subsequence.

A recombinant protein can be produced by “recombinant means”, which can refer to techniques where proteins are isolated, the cDNA sequence coding the protein identified and inserted into an expression vector. The vector is then introduced into a cell and the cell expresses the protein. Recombinant means also encompasses the ligation of coding or promoter DNA from different sources into one vector for expression of a PPC, constitutive expression of a protein, or inducible expression of a protein.

Polynucleotide sequences encoding one or more recombinant serine protease inhibitor polypeptide can be operatively linked to expression control sequences (e.g., a promoter). An expression control sequence operatively linked to a coding sequence is ligated such that expression of the coding sequence is achieved under conditions compatible with the expression control sequences. The expression control sequences include, but are not limited to appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

The polynucleotide sequences encoding one or more recombinant serine protease inhibitor polypeptides can be inserted into an expression vector including, but not limited to a plasmid, virus or other vehicle that can be manipulated to allow insertion or incorporation of sequences and can be expressed in prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors that can express and replicate in a host are known in the art.

In an aspect, a composition disclosed herein comprises nucleic acid molecules that encode the recombinant serine protease inhibitor-derived peptides or fragments thereof disclosed herein in an expression construct or in a single or separate cassette. Disclosed herein is an expression construct that can express recombinant serine protease inhibitor-derived peptides or fragments thereof.

A disclosed expression cassette can include 5′ and 3′ regulatory sequences operably linked to a polynucleotide disclosed herein. “Operably linked” is intended to mean a functional linkage between two or more elements. For example, an operable linkage between a polynucleotide disclosed herein and a regulatory sequence (e.g., a promoter) is a functional link that allows for expression of a polynucleotide disclosed herein. Operably linked elements can be contiguous or non-contiguous. When used to refer to the joining of two protein coding regions, by operably linked is intended that the coding regions are in the same reading frame. An expression cassette can further comprise at least one additional polynucleotide to be co-transformed into the organism. Alternatively, one or more polypeptide(s) can be expressed on one or more expression cassettes. Expression cassettes can be provided with a plurality of restriction sites and/or recombination sites for insertion of the polynucleotide to be under the transcriptional regulation of the regulatory regions.

The regulatory regions (i.e., promoters, transcriptional regulatory regions, and translational termination regions) and/or the polynucleotides disclosed herein can be native/analogous to the host cell or to each other. Alternatively, the regulatory regions and/or the polynucleotide employed in the invention can be heterologous to the host cell or to each other. As used herein, “heterologous” in reference to a sequence can refer to a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

In preparing the expression cassette, the various DNA fragments can be manipulated, so as to provide for the DNA sequences in the proper orientation and, as appropriate, in the proper reading frame. Toward this end, adapters or linkers can be employed to join the DNA fragments or other manipulations can be involved to provide for convenient restriction sites, removal of superfluous DNA, removal of restriction sites, or the like. For this purpose, in vitro mutagenesis, primer repair, restriction, annealing, resubstitutions, e.g., transitions and transversions, can be involved.

A number of promoters can be used in the practice of the invention. The promoters can be selected based on the outcome. The choice of promoters depends on several factors including but not limited to efficiency, selectability, inducibility, expression level, and cell- or tissue-preferential expression. The nucleic acids can be combined with constitutive, tissue-preferred, inducible, or other promoters for expression in the host organism. One skilled in the art can appropriately select and position promoters and other regulatory regions relative to the coding sequence.

### Wound Healing Formulations

Aspects of the invention are directed towards compositions and formulations for promoting wound healing. For example, such formulations can comprise recombinant polypeptides or fragments thereof as described herein.

A “formulation” can refer to a composition containing at least one active therapeutic agent or pharmaceutical, and one or more carriers, excipients, or diluent. For example, a “carrier,” “pharmaceutically acceptable carrier”, “excipient”, and/or “diluent” can be used interchangeably, and can refer to any liquid, gel, paste, salve, solvent, fluid ointment base, suspension, spray, liposome, micelle, giant micelle, and the like, which is suitable for use in contact with living animal or human tissue without causing adverse physiological responses, and which does not interact with the other components of the composition in a deleterious manner. For example, a number of carrier ingredients are known for preparing topical formulations, including but not limited to gelatin, polymers, fats and oils, lecithin, collagens, alcohols, and water.

In certain embodiments, the formulation can be administered to a subject in a therapeutically effective amount. An “effective amount” or “therapeutically effective amount” can refer to an amount of the formulation that is sufficient to produce a therapeutic and/or beneficial effect. The effective amount will vary with the age, general condition of the subject, the severity of the condition being treated, the agent administered, the duration of the treatment, the nature of any concurrent treatment, the pharmaceutically acceptable carrier or excipient used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)).

In embodiments, a therapeutically effective amount can be between about 0.1 μg/kg and about 100 mg/kg. For example, the therapeutically effective amount can be about 0.1 μg/kg, about 1 μg/kg, about 10 μg/kg, about 100 μg/kg, about 1 mg/kg, about 10 mg/kg, or about 100 mg/kg. In embodiments, the therapeutically effective amount is greater than about 100 mg/kg.

In embodiments, a therapeutically effective amount can be between about 0.01 mg/ml and about 500 mg/ml. For example, a therapeutically effective amount can be about 0.01 mg/ml, about 0.1 mg/ml, about 1 mg/ml, about 10 mg/ml, about 100 mg/ml, about 200 mg/ml, about 300 mg/ml, about 400 mg/ml, about 500 mg/ml, or greater than 500 mg/ml.

In embodiments, a therapeutically effective amount of the formulation can be administered, once a day, twice a day, three times a day, or as needed. In other embodiments, the therapeutically effective amount of the formulation can be administered every other day, every three days, once a week, or every other week, or monthly.

Embodiments of the disclosure provide forumulations prepared for the administration of the active agent(s) to a subject (e.g., a human) using any available method and route suitable for drug delivery, including in vivo and ex vivo methods, as well as systemic and localized routes of administration. Routes of administration include intranasal, intramuscular, intratracheal, subcutaneous, intradermal, intravitreal, topical application, intravenous, rectal, nasal, oral, and other enteral and parenteral routes of administration. Routes of administration can be combined or adjusted depending upon the agent and/or the target effect. An active agent can be administered in a single dose or in multiple doses.

For example, wound healing formulation can be prepared for topical administration. Such formulations can be referred to as “topical formulations”. Such topical formulation can promote wound healing in a mammalian subject when topically administered. For example, a “topical formulation” can refer to a composition containing at least one active therapeutic agent or pharmaceutical, including an excipient, in which the therapeutic agent or pharmaceutical can be placed for direct application to a skin surface and from which an effective amount of therapeutic agent or pharmaceutical is released. Examples of topical formulations include but are not limited to ointment, cream, lotion, suspension, aqueous solution, dispersion, salve, gel, spray, film, or paste.

Formulations as described herein can further comprises viscosity increasing agents, ointment bases (e.g., cream bases), antimicrobial preservatives, temperature and pH sensing probes, emulsifying agents, and/or solvents.

A “viscosity increasing agent” can refer to an agent that is used to thicken a formulation. Exemplary viscosity increasing agents can include, for example, cetostearyl alcohol, cholesterol, stearyl alcohol, chlorocresol, white wax, stearic acid, cetyl alcohol, or a combination thereof. The viscosity increasing agent can be in the topical formation at a concentration of about 1.0-10% (w/w). For example, the topical formulation can comprise about 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, or 9.5-10% (w/w) of the viscosity increasing agent. Alternatively, the topical formulation can comprise about 1-5%, 2.5-7.5%, or 5-10% (w/w) of the viscosity increasing agent.

An “ointment base” can be any semisolid preparation or vehicle into which an active agent can be incorporated. Exemplary ointment bases include, but are not limited to, oleaginous ointment bases (e.g., white petrolatum or white ointment), absorption ointment bases (e.g., hydrophilic petrolatum, anhydrous lanolin, Aquabase™, Aquaphor®, and Polysorb®), water/oil emulsion ointment bases (e.g., cold cream, hydrous lanolin, rose water ointment, Hydrocream™, Eucerin®, and Nivea®), oil/water emulsion ointment bases (e.g., hydrophilic ointments, Dermabase™, Velvachol®, and Unibase®), and water-miscible ointment bases (e.g., polyethylene glycol (PEG) ointment and Polybase™). Ointment bases can be pharmacologically inert but can entrap water in order to provide an emollient protective film. In an embodiment, the ointment base can be any petrolatum compound (e.g., petrolatum, white petrolatum, white soft paraffin, liquid petrolatum, liquid paraffin). In a further specific embodiment, the ointment base is white petrolatum (CAS number 8009 Mar. 8). The ointment base can be in the topical formation at a concentration of about 5-30% (w/w), e.g., 10-30% (w/w). For example, the topical formulation can comprise about 5-25%, 5-20%, 5-15%, 5-15%, 10-15%, 15-20%, 20-25%, or 25-30% (w/w) of the ointment base. For example, the topical formulation can comprise about 5, 6, 7, 8, 9, 10, 11, 12, 13, 14, 15, 16, 17, 18, 19, 20, 21, 22, 23, 24, 25, 26, 27, 28, 29, or 30 percent (w/w) of the ointment base.

In some embodiments, the “ointment base” described herein contains less than 20% water and volatiles, and more than 50% hydrocarbons, waxes, or polyols as the vehicle.

In some embodiments, the “ointment base” described herein is a “cream base,” which contains more than 20% water and volatiles and/or can contain less than 50% hydrocarbons, waxes, or polyols as the vehicle for the drug substance. The cream base can be a multiphase preparation containing a lipophilic phase and an aqueous phase. In some instances, the cream base is a lipophilic cream base, which has a lipophilic phase as the continuous phase. Such a cream base can contain water-in-oil emulsifying agents such as wool alcohols, sorbitan esters and monoglycerides. In other instances, the cream base is a hydrophilic cream base, which has an aqueous phase as the continuous phase. Such a cream base can contain oil-in-water emulsifying agents such as sodium or trolamine soaps, sulfated fatty alcohols, polysorbates and polyoxyl fatty acid and fatty alcohol esters, which can be in combination with water-in-oil emulsifying agents, if needed.

As used herein, the term “aqueous solution” can refer to a solution, wherein at least one solvent is water and the weight % of water in the mixture of solvents is at least 50%, at least 60%, at least 70% or at least 90%. In some embodiments, aqueous solution is a solution in which water is the only solvent. In some embodiments, aqueous solution is a buffer (e.g., phosphate buffer or a carbonate buffer). In some embodiments, the buffer is physiological buffer or a pharmaceutically acceptable buffer. In some embodiments, the buffer is any one of buffers described, for example, in Y.-C. Lee et al. International Journal of Pharmaceutics 253 (2003) 111-119, the disclosure of which is incorporated herein by reference in its entirety. In some embodiments, the buffer comprises maleic acid, tartaric acid, lactic acid, citric acid, acetic acid, sodium bicarbonate, sodium phosphate, or mixtures thereof. In some embodiments, the pH range of the buffer is from about 3 to about 9, from about 4 to about 8, from about 5 to about 7, from about 6 to about 7, from about 3 to about 5, from about 3 to about 7, from about 4 to about 6, or from about 6 to about 6. In some embodiments, the pH of the buffer is about 4, about 5, about 6, about 6.4, about 6.5, about 6.6, about 7, about 7.5, or about 8.

An “antimicrobial preservative” can be any compound that can destroy microbes, prevent the multiplication or growth of microbes, or prevent the pathogenic action of microbes. Exemplary antimicrobial preservatives include, but are not limited to, a paraben compound (an ester of para-hydroxybenzoic acid; e.g., paraben, methylparaben, ethylparaben, propylparaben, butylparaben, heptylparaben, benzylparaben, isobutylparaben, isopropylparaben, benzylparaben, or their sodium salts), benzalkonium chloride, benzethonium chloride, benzyl alcohol, boric acid, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. The antimicrobial preservative can be present in the topical formation at a concentration of about 0.005-0.2%, e.g., about 0.01-0.2% (w/w). For example, the topical formulation can comprise about 0.005-0.01%, 0.01-0.05%, 0.05-0.1%, 0.1-0.15%, or 0.15-0.2% (w/w) of the antimicrobial preservative. For example, the topical formulation can comprise about 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, or 0.2 percent (w/w) of the antimicrobial preservative.

An “emulsifying agent” can refer to a compound or substance which acts as a stabilizer for a mixture of two or more liquids that are normally immiscible (unmixable or unblendable). Exemplary emulsifying agents can include, but are not limited to, natural emulsifying agents (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and Veegum [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, propylene glycol monostearate, and polyvinyl alcohol), carbomers (e.g., carboxy polymethylene, polyacrybc acid, acrylic acid polymer, and carboxy vinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, and methylcellulose), sorbitan fatty acid esters (e.g., polyoxyethylene sorbitan monolaurate [Tween® 20], polyoxyethylene sorbitan [Tween® 60], polyoxyethylene sorbitan monooleate [Tween® 80], sorbitan monopalmitate [Span® 40], sorbitan monostearate [Span® 60], sorbitan tristearate [Span® 65], glyceryl monooleate, and sorbitan monooleate [Span® 80]), polyoxyethylene esters (e.g., polyoxyethylene monostearate [Myq® 45], polyoxyethylene hydrogenated castor oil, polyethoxylated castor oil, polyoxymethylene stearate, and Solutol), sucrose fatty acid esters, polyethylene glycol fatty acid esters (e.g., Cremophor®), polyoxyethylene ethers (e.g., polyoxyethylene lauryl ether [Brij® 30]), and poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, and docusate sodium, and/or combinations thereof. The emulsifying agent can be present in the topical formation at a concentration of about 0.5-10% (w/w), e.g., 0.5-6% (w/w). For example, the topical formulation can comprise about 0.5-1%, 1-1.5%, 1.5-2%, 2-2.5%, 2.5-3%, 3-3.5%, 3.5-4%, 4-4.5%, 4.5-5%, 5-5.5%, 5.5-6%, 5-10%, 6-10%, or 8-10% (w/w) of the emulsifying agent. For example, the topical formulation can comprise about 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 percent (w/w) of the emulsifying agent.

Formulations as described herein, such as topical formulations, can further contain one or more solvents (e.g., non-water solvents or water). Exemplary non-water solvents can include, but are not limited to, any known solvent including propylene glycol, glycol, and mixtures thereof. The non-water solvent can be present in the topical formation at a concentration of about 2-65% (w/w). For example, the topical formulation can comprise about 2-15%, 15-30%, 30-45%, or 45-65% (w/w) of the solvent. In some embodiments, the topical formulation of the invention can also contain water.

In embodiments, formulations as described herein, such as topical formulations, can further comprise one or more emollients, fragrances, or pigments.

The formulation can also be used in conjunction with a wound dressing (e.g., bandage with adhesive, plaster patch and the like) (e.g., cyclohexane, n-hexane, n-decane, i-octane, octane, butyl ether, carbon tetrachloride, triethyl amine, i-propyl ether, toluene, p-xylene, t-butyl methyl ether, benzene, benzyl ether, dichloromethane, methylene chloride, chloroform, dichloroethane, ethylene di chloride, 1-butanol, i-butyl alcohol, tetrahydrofuran, ethyl acetate, 1-propanol, 2-propanol, methyl acetate, cyclohexanone, methyl ethyl ketone (MEK), nitrobenzene, benzonitrile, 1,4-dioxane, or p-dioxane). In certain embodiments, the formulation includes a hydrogel.

In embodiments, the formulation can further comprise one or more additional active ingredients. For example, the one or more additional active ingredients comprises an antibiotic, a pain reliever, an anti-inflammatory, an anti-scarring agent, a moisturizer, a steroid, an immune modulator, or a growth factor. Non-limiting examples of the active ingredient comprise human serum albumen, calcium, bovine thrombin, human Thrombin (hThrombin), rhThrombin, factor Vila, factor XIII, recombinant Factor XIII (rF actor XIII), thromboxane A2, prostaglandin-2a, epidermal growth factor, platelet derived growth factor, Von Willebrand factor, tumor necrosis factor (TNF), TNF-alpha, transforming growth factor (TGF), TGF-alpha, TGF-beta, insulin like growth factor, fibroblast growth factor, keratinocyte growth factor, nerve growth factor.

“Antibiotic” can refer to a substance that controls the growth of bacteria, fungi, or similar microorganisms, wherein the substance can be a natural substance produced by bacteria or fungi, or a chemically/biochemically synthesized substance (which can be an analog of a natural substance), or a chemically modified form of a natural substance. Any antibiotic can be used with the disclosed composition or methods. Examples of antibiotics that can be used include but are not limited to antimicrobial peptides (AMP), aminoglycosides (such as amikacin, gentamicin, kanamycin, neomycin, netilmicin, streptomycin, tobramycin, and paromomycin): ansamycins (such as geldanamycin, and herbimycin); carbacephems (such as loracarbef, ertapenem, doripenem, imipenem/cilastatin, and meropenem): cephalosporins (such as cefadroxil, cefazobn, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, and ceftobiprole): gly copeptides (such as teicoplanin and vancomycin); macrolides (such as azithromycin, clarithromycin, dirithromycin, erythromycin, roxithromycin, troleandomycin, telithromycin, and spectinomycin): monobactams (such as aztreonam); penicillins (such as amoxicillin, ampicillin, azlocillin, carbenicillin, cloxacillin, dicloxacillin, flucloxacillin, mezlocillin, meticillin, amoxycillin, clavamox, clavulanic acid, nafcillin, oxacillin, penicillin, piperacillin, and ticarcillin): peptides (such as bacitracin, colistin, and polymyxin b): quinolones (such as ciprofloxacin, enoxacin, gatifloxacin, levofloxacin, lomefloxacin, moxifloxacin, norfloxacin, ofloxacin, trovafloxacin, grepafloxacin, and sparfloxacin); sulfonamides (such as mafenide, prontosil (archaic), sulfacetamide, sulfamethizole, sulfanilimide (archaic), sulfasalazine, sulftsoxazole, trimethoprim, and trimethoprim-sulfamethoxazole); tetracyclines (such as demeclocycline, doxycycline, minocycline, oxy tetracycline, and tetracycline); and others (such as arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampicin, thiamphenicol, and tinidazole) or combinations thereof.

Other active ingredients which can be included in a formulation, such as that described herein, include a pain reliever, an anti-inflammatory, an anti-scarring agent, a moisturizer, a steroid, an immune modulator, or a growth factor.

For example, the term “pain reliever” or “pain relieving agent” can refer to one having an action of relieving pain. Non-limiting examples of pain relievers, can include acetaminophen, ibuprofen, ketoprofen, diclofenac, naproxen, aspirin, and combinations thereof, as well as prescription analgesics, non-limiting examples of which include propyxhene HCl, codeine, mepridine, and combinations thereof.

For example, “immune modulator” can refer to a substance that can alter (e.g., inhibit, decrease, increase, enhance or stimulate) the working of any component of the innate, humoral or cellular immune system of a mammal. For example, the “immune modulator” can be SERP-1, or other immune modulators derived from natural sources.

For example, the term “growth factor” can refer to proteins that promote growth, and include, for example, hepatic growth factor; fibroblast growth factor; vascular endothelial growth factor; nerve growth factors such as NGF-β; platelet-derived growth factor; transforming growth factors (TGFs) such as TGF-α and TGF-β; insulin-like growth factor-I and -II; erythropoietin (EPO); osteoinductive factors; interferons such as interferon-α, -β, and -γ; and colony stimulating factors (CSFs) such as macrophage-CSF (M-CSF), granulocyte-macrophage-CSF (GM-CSF), and granulocyte-CSF (G-CSF). As used herein, the term growth factor includes proteins from natural sources or from recombinant cell culture and biologically active equivalents of the native-sequence growth factor, including synthetically produced small-molecule entities and pharmaceutically acceptable derivatives and salts thereof.

It can be desirable to control recombinant serine protease inhibitor protein release in order to potentially extend the release time (i.e., delayed release) and increase stability for a long-term topical treatment, such as wound healing. As such, embodiments described herein comprise recombinant polypeptides or fragments thereof with hydrogels to form slow release composition. Thus, embodiments are also directed to hydrogels that include a recombinant polypeptides or fragments thereof, or nucleic acid encoding a recombinant polypeptides or fragments thereof, and optionally, other active ingredients as discussed herein. In some examples the hydrogels are incorporated into a wound dressing for promoting wound healing. To this end, aspects of the disclosure are directed to wound dressings, and methods of using such wound dressings.

A “hydrogel” can refer to a substance formed when an organic polymer (natural or synthetic) is cross-linked via covalent, ionic, or hydrogen bonds to create a three-dimensional open-lattice structure which entraps water molecules to form a gel. Non-limiting examples of materials which can be used to form a hydrogel include polysaccharides such as alginate, chitosan, polyphosphazenes, and polyacrylates such as polyhydroxyethyl methacrylate (poly-HEMA) and poly-N-(2-hydroxypropyl) methacrylamide (poly-HPMA), which are crosslinked ionically, or block copolymers such as PLURONICS™ (BASF Corporation) or TETRONICS™ (BASF Corporation), polyethylene oxide-polypropylene glycol block copolymers which are crosslinked by temperature or pH sensing probes, respectively. Other materials include proteins such as fibrin, polymers such as polyvinylpyrrolidone, hyaluronic acid and collagen.

The hydrogel can also include gelatin, cellulose, or collagen-based materials. In some examples, the gelatin-based substrate includes an absorbable sponge, powder or film of cross-linked gelatin, for example, GELFOAM® (Upjohn, Inc., Kalamazoo, Mich.) which is formed from denatured collagen. A cellulose-based substrate includes an appropriate absorbable cellulose such as regenerated oxidized cellulose sheet material, for example, SURGICEL® (Johnson & Johnson, New Brunswick, N.J.) or Oxycel® (Becton Dickinson, Franklin Lakes, N.J.). Collagen materials can include an appropriate resorbable collagen, such as purified bovine corium collagen, for example, AVITENE® (MedChem, Wobum, Mass.), HELISTAT® (Marion Merrell Dow, Kansas City, Mo.), HEMOTENE® (Astra, Westborough, Mass.), or SURGIFOAM® (Johnson & Johnson, New Brunswick, NJ). See also, chitosan bandages for wound healing, such as HemCon®, Tricol Biomedical Inc.

Chitosan-based hydrogels, such as chitosan-collagen hydrogel, have also been tested for wound treatment for delivery of antimicrobials, peptides, and growth factors showing significant promotion on wound healing (Liu et al, RSC Adv. (2018), Elviri et al, Expert Opin. Drug Deliv. (2017), Hamedi et al, Carbohydr. Polym. (2018), Riva et al., Adv. Polym. Sci. (2011), Liu et al, Adv. Polym. Sci. (2011)). Considering the biocompatible, antimicrobial, biologically adhesive, hemostatic effect and applications for drug delivery, a chitosan-based hydrogel as a drug delivery system for the treatment of wound healing with recombinant serine protease inhibitor polypeptides or fragments thereof are disclosed herein. In addition to the discovery of function of recombinant polypeptides or fragments thereof in promoting wound healing as described herein, a chitosan-collagen hydrogel carrier can efficiently deliver recombinant serine protease inhibitor polypeptides locally to a wound site and promote healing. Thus, in some embodiments, a recombinant polypeptide or fragment thereof, or nucleic acid encoding a recombinant polypeptide or fragment thereof (and other active ingredients as described herein) are incorporated into a chitosan-collagen hydrogel carrier.

During wound healing, collagen accumulation and organization are correlated with scar formation. Collagens play a crucial role in angiogenesis during tissue regeneration. Collagen I is a central factor allowing for endothelial cells to initiate precapillary cord formation. In contrast increased deposition of collagen III reduces the density of blood vessels at sites of wound healing (Davis and Senger et al., Circ. Res. (2005), O'Rourke et al, Adv. Wound Care. (2018)). In embodiments, the wound dressing that includes a recombinant polypeptide, fragment thereof, or nucleic acid encoding a recombinant polypeptide or fragment thereof can be formed of a biomaterial, such as poly [b-(1-4)-2-amino-2-deoxy-D-glucopyranose], which can be referred to as chitosan, and, in embodiments, in combination with collagen, e.g., collagen-chitosan hydrogels. The wound dressing can be formed into a sponge-like or woven configuration via the use of an intermediate structure or form producing steps. The biomaterial comprises an interconnected open porous structure, and/or an oriented open lamella structure, and/or an open tubular structure, and/or an open honeycomb structure, and/or a filamentous structure.

In another embodiment, the formulation as described herein can be delivered in a controlled release formulation and/or sustained release matrix. As used herein, a sustained-release matrix can refer to a matrix made of materials, for example polymers, which are degradable by enzymatic or acid-based hydrolysis or by dissolution. Once inserted into the body, the matrix is acted upon by enzymes and body fluids. A sustained-release matrix can be chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho) esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the formulations as described herein can be formed by impregnation of the composition or pharmaceutical composition described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the embodiments described herein.

Aspects of the invention are also directed towards a bandage, wound dressing, or graft permeated with a recombinant polypeptide or fragment thereof for in vivo use. The phrase “in vivo use” can refer to a use wherein the recombinant polypeptide or fragment thereof permeated graft is at least partially positioned on or within the body of a subject. For example, use of a recombinant polypeptide permeated graft placed on a wound of a subject to facilitate wound healing can be considered an in vivo use. Similarly, use of a recombinant polypeptide permeated graft implanted within a subject following a surgical procedure to facilitate tissue regeneration can be considered an in vivo use.

The bandage, wound dressing, or graft can comprise a bioscaffold permeated with recombinant polypeptide or fragment thereof. As used herein, “bioscaffold” can refer to a substrate on which cells can grow. In embodiments, the bioscaffold can mimic the native biological extracellular matrix of the tissue it is meant to regenerate.

In an embodiment, the bandage, wound dressing, or graft can comprise a hydrogel. A hydrogel is a three-dimensional solid that comprises a network of hydrophilic polymer chains that results from the hydrophilic polymer chains being held together by cross-links. Because of the inherent cross-links, the structural integrity of the hydrogel network does not dissolve from the high concentration of water. Hydrogels are highly absorbent (they can contain over 90% water) natural or synthetic polymeric networks. Hydrogels also possess a degree of flexibility very similar to natural tissue, due to their significant water content. Biohydrogels are known in the art, and have been developed for a broad scope of therapeutic applications, such as for the release of Biomacromolecules or drugs, wound healing, or as a barrier for contact lenses or ocular surface injuries. See, for example, Mateescu, Mihaela, et al. “Antibacterial peptide-based gel for prevention of medical implanted-device infection.” PLOS One10.12 (2015): e0145143; Zhao, Fan, Man Lung Ma, and Bing Xu. “Molecular hydrogels of therapeutic agents.” Chemical Society Reviews 38.4 (2009): 883-891; each of which are incorporated herein by reference in their entireties.

In embodiments, the bandage, wound dressing, or graft can comprise a “biodegradable polymer”, which can refer to a polymer which can be broken down into organic substances, such as by living organisms. For example, the bandage, wound dressing, or graft can comprise biodegradable polymers such as chitosan, collagen, fibrin, silk fibroin, carboxylated silk fibroin, silk sericin polyarginine, polylysine, alginate, cyanoacrylate, dermabond, and the like, and combinations thereof. For example, a bandage, wound dressing, or graft can partially or completely comprise one or more biodegradable polymers.

In embodiments, the polymer can be a natural polymer or a synthetic polymer. Natural polymers occur in nature and can be extracted, such as polysaccharides or proteins. Non-limiting examples of polysaccharides comprise chondroitin sulfate, heparin, heparan, alginic acid (i.e., alginate), hyaluronic acid, dermatan, dermatan sulfate, pectin, carboxymethyl cellulose, chitosan, melanin (and its derivatives, such as eumelanin, pheomelanin, and neuromelanin), agar, agarose, gellan, gum, and the like as well as their salt forms (such as sodium salt and potassium salt). Non-limiting examples of proteins comprise collagen, alkaline gelatin, acidic gelatin, gene recombination gelatin, and so on.

Synthetic polymers are man-made molecules formed by the polymerization of a variety of monomers, such as macromolecules comprising polyacrylic acid, polyaspartic acid, polytartaric acid, polyglutamic acid, polyfumaric acid, polyarginine, polylysine, polyhistidine, and so on as well as their salt forms (such as sodium salt and potassium salt). Non-limiting examples of synthetic polymers comprise cyanoacrylate, pluronic diacrylate, amino acid-based poly(ester amide) polymers (such as those based on arginine, lysine, or histidine).

In embodiments, the polymer can be a cationic polymer, which can refer to a polymer with a positive charge. For example, the graft comprises cationic polymers such as polyarginine, polylysine, or polyhistidine, and the like. The skilled artisan will recognize that the polymer can be any polymer that is suitable to form electrostatic nanocomplexes with negatively charged compounds or neutral compounds.

### Methods for Promoting Wound Healing

Any of the formulations described herein can be used for treating and/or promoting wound healing in a subject in need of the treatment. As used herein, the terms “treat,” “treating” or “treatment” can refer to any type of action that imparts a modulating effect, which, for example, can be a beneficial and/or therapeutic effect, to a subject afflicted with a condition, disorder, disease or illness, including, for example, improvement in the condition of the subject (e.g., in one or more symptoms), delay in the progression of the disorder, disease or illness, delay of the onset of the disease, disorder, or illness, and/or change in clinical parameters of the condition, disorder, disease or illness, etc., as can be well known in the art.

The term “wound” can refer to an injury to living tissue caused by a cut, blow, or other impact (e.g., caused by a medical condition such as a skin disorder), such as one in which the skin is cut or broken. Any disruption of normal anatomy, from whatever cause, can be considered a wound. Causes of wounds can include but are not limited to traumatic injuries such as mechanical, thermal, and incisional injuries; elective injuries such as surgery and resultant incisional hernias: acute wounds, chronic wounds, infected wounds, dermal wounds, and sterile wounds, as well as wounds associated with disease states (i.e. ulcers caused by diabetic neuropathy: skin disorders).

Wounds which can benefit from embodiments as described herein include but are not limited to cuts and lacerations, surgical incisions or wounds, punctures, grazes, scratches, compression wounds, abrasions, friction wounds (e.g. nappy rash, friction blisters), decubitus ulcers (e.g. pressure or bed sores); thermal effect wounds (burns from cold and heat sources, directly or through conduction, convection, or radiation, and electrical sources), chemical wounds (e.g. acid or alkali burns) or pathogenic infections (e.g. viral, bacterial or fungal) including open or intact boils, skin eruptions, blemishes and acne, ulcers, chronic wounds, (including diabetic-associated wounds such as lower leg and foot ulcers, venous leg ulcers and pressure sores), skin graft/transplant donor and recipient sites, immune response conditions, e.g. psoriasis and eczema, stomach or intestinal ulcers, oral wounds, including a ulcers of the mouth, damaged cartilage or bone, amputation wounds and corneal lesions.

In embodiments, the wound can be a transplant wound. In embodiments, the wound can not be a transplant wound.

For example, the wound can comprise a burn wound, a surgical wound, a diabetic ulcer, a pressure ulcer, an ischemic wound, a venous and/or arterial ulcer, or a chronic wound.

A wound is dynamic and the process of healing is a continuum requiring a series of integrated and interrelated cellular processes that begin at the time of wounding and proceed beyond initial wound closure through arrival at a stable scar. These cellular processes are mediated or modulated by humoral substances including but not limited to cytokines, lymphokines, growth factors, and hormones.

The term “wound healing” can refer to the dynamic and complex process of replacing devitalized or missing cellular structures and/or tissue layers. In embodiments, wound healing can refer to improving, by some form of intervention, the natural cellular processes and humoral substances such that healing is faster, and/or the resulting healed area has less scaring and/or the wounded area possesses tissue tensile strength that is closer to that of uninjured tissue.

The term “promotion of wound healing” or “promoting wound healing” can refer to the inducement of an increased level or rate of replacement for devitalized or missing cellular structures and/or tissue layers. As an example, promotion of wound healing can be indicated by partial or complete ulcer closure or an increase in the healing rate of an ulcer (including but not limited to more rapid changes in ulcer size, area, or severity, a more rapid closure of the ulcer, and/or an increase in the percentage change from baseline in ulcer size, area, or severity when compared to a control ulcer treated with a placebo).

As used herein, the term “dermal wound” can refer to an injury to the skin in which the skin is cut or broken.

In embodiments, the wound can be any internal wound, e.g., where the external structural integrity of the skin is maintained, such as in bruising or internal ulceration, or external wounds, for example cutaneous wounds, and consequently the tissue can be any internal or external bodily tissue. In one embodiment the tissue is skin (such as human skin), i.e. the wound is a cutaneous wound, such as a dermal or epidermal wound.

The human skin is composed of two distinct layers, the epidermis and the dermis, below which lies the subcutaneous tissue. The primary functions of the skin are to provide protection to the internal organs and tissues from external trauma and pathogenic infection, sensation and thermoregulation.

The outermost layer of skin, the epidermis, is approximately 0.04 mm thick, is avascular, is comprised of four cell types (keratinocytes, melanocytes, Langerhans cells, and Merkel cells), and is stratified into several epithelial cell layers. The inner-most epithelial layer of the epidermis is the basement membrane, which is in direct contact with, and anchors the epidermis to, the dermis. All epithelial cell division occurring in skin takes place at the basement membrane. After cell division, the epithelial cells migrate towards the outer surface of the epidermis. During this migration, the cells undergo a process known as keratinization, whereby nuclei are lost and the cells are transformed into tough, flat, resistant non-living cells. Migration is completed when the cells reach the outermost epidermal structure, the stratum corneum, a dry, waterproof squamous cell layer which helps to prevent desiccation of the underlying tissue. This layer of dead epithelial cells is continuously being sloughed off and replaced by keratinized cells moving to the surface from the basement membrane. Because the epidermal epithelium is avascular, the basement membrane is dependent upon the dermis for its nutrient supply.

The dermis is a highly vascularized tissue layer supplying nutrients to the epidermis. In addition, the dermis contains nerve endings, lymphatics, collagen protein, and connective tissue. The dermis is approximately 0.5 mm thick and is composed predominantly of fibroblasts and macrophages. These cell types are largely responsible for the production and maintenance of collagen, the protein found in all animal-connective tissue, including the skin. Collagen is primarily responsible for the skin's resilient, elastic nature. The subcutaneous tissue, found beneath the collagen-rich dermis, provides for skin mobility, insulation, calorie storage, and blood to the tissues above it.

Wounds can be classified in one of two general categories, partial thickness wounds or full thickness wounds. A partial thickness wound is limited to the epidermis and superficial dermis with no damage to the dermal blood vessels. A full thickness wound involves disruption of the dermis and extends to deeper tissue layers, involving disruption of the dermal blood vessels. The healing of the partial thickness wound occurs by simple regeneration of epithelial tissue. Wound healing in full thickness wounds is more complex. Cutaneous wounds described herein can be partial thickness or full thickness wounds.

The term “chronic wound” can refer to a wound that has not healed. For example, a wound that does not heal within 1 month, 2 months, 3 months, or longer than 3 months is considered chronic. Chronic wounds, including pressure sores, venous leg ulcers and diabetic foot ulcers, can simply be described as wounds that fail to heal. Whilst the exact molecular pathogenesis of chronic wounds is not fully understood, it is acknowledged to be multi-factorial. As the normal responses of resident and migratory cells during acute injury become impaired, these wounds are characterized by a prolonged inflammatory response, defective wound extracellular matrix (ECM) remodeling and a failure of re-epithelialization.

An “infected wound” can refer to a wound in which bacteria and/or other microorganisms are grown and infiltrated in the wound part. Infected wounds are conditions that have obvious signs of inflammation and delay healing.

A “burn wound” can refer to a case where a large surface area of an individual's skin has been removed or lost due to heat and/or chemical agents.

The term “ulcer” can refer to a lesion through the skin or a mucous membrane resulting from loss of tissue, for example with inflammation. Non-limiting examples of ulcers include acute decubitus ulcer (i.e., severe form of bedsore), anastomotic ulcer, Buruli ulcer, chrome ulcer, chronic ulcer, stress ulcer, decubitus ulcer, dendritic corneal ulcer, dental ulcer, diphtheritic ulcer, distention ulcer, elusive ulcer, fascicular ulcer, Fenwick-Hunner ulcer, Gaboon ulcer, gastric ulcer, gravitational ulcer, gummatous ulcer, healed ulcer, herpetic ulcer, Hunner ulcer, hypopyon ulcer, indolent ulcer, inflamed ulcer, Mann-Williamson ulcer, marginal ring ulcer of the cornea, Marjolin ulcer, Meleney ulcer, Mooren ulcer, Oriental ulcer, penetrating ulcer, peptic ulcer, perforated ulcer, perforating ulcer of foot, phagedenic ulcer, pressure ulcer, recurrent aphthous ulcers, ring ulcer of the cornea, rodent ulcer, Saemisch ulcer, serpent ulcer of the cornea, sepiginous ulcer, serpiginous corneal ulcer, simple ulcer, sloughing ulcer, soft ulcer, stasis ulcer, stercoral ulcer, stomal ulcer, Curling ulcer, Sutton ulcer, syphilitic ulcer, Syriac ulcer, tanner's ulcer, trophic ulcer, tropical ulcer, undermining ulcer, varicose ulcer, venereal ulcer, venous ulcer, and Zambesi ulcer.

Diabetes can cause wound to heal more slowly, thereby increasing the risk that people with diabetes will develops infections. The term “diabetic wound” refers to any wound in an individual having diabetes, including chronic wounds occurring in diabetic patients.

The formulation as described herein can be applied to a wound site following a suitable dosage and treatment regimen. The dosage and administration regimen for the described method will depend on the nature and condition of the wound being treated, the age and condition of the patient, and any prior or concurrent therapy. In some instances, the formulation can be applied once every week, once every other day, once daily, twice daily, three times daily, or four time daily for a suitable period of time. The treatment can be terminated when the wound is recovered. When necessary, the treatment can resume, for example, if a wound recurs.

The subject to be treated by the formulation can be a human or a non-human mammal. As used herein, the term “subject” and “patient” are used interchangeably herein and can refer to both human and nonhuman animals. The term “nonhuman animals” of_the disclosure includes vertebrates, e.g., mammals and non-mammals, such as nonhuman primates, sheep, dog, cat, horse, cow, rodents (e.g., mice, rats, etc.) and the like. For example, the subject is a human patient. In embodiments, the subject of this disclosure is a human subject. A “subject in need thereof’ or “a subject in need of’ is a subject known to have, or is suspected of having a surface wound, such as a wound in the skin and surrounding tissue.

In some embodiments, the subject is a human patient having an open wound, which can refer to an injury or damage to living tissues (e.g., skin) that cause a disruption in the normal continuity of biological structures. An open wound can include, but is not limited to, an abrasion, incision, laceration, puncture, avulsion, cut, or other similar injuries.

In other embodiments, the subject is a human patient having a chronic wound, which can be injuries or damage to living tissues (e.g., skin) that cause a disruption in the normal continuity of biological structures and do not heal in an orderly set of stages and/or in a predictable amount of time. A chronic wound can include, but is not limited to a surgical wound, a traumatic wound, a pressure ulcer, a venous ulcer, or a diabetic ulcer. In other examples, a chronic wound can be associated with a disease or disorder, for example, a carcinoma, burn, bedsore, a skin disorder such as atopic dermatitis.

In one example, the subject is a human patient having an ulcer, such as a foot ulcer, associated with diabetes (e.g., type I or type II). Diabetes mellitus (also known as diabetes) is a group of metabolic diseases which result in high blood sugar levels over a prolonged period. Diabetes can result from the pancreas not producing enough insulin or the cells of the body not responding properly to the insulin produced. The three main types of diabetes mellitus are Type I (also known as “insulin-dependent diabetes mellitus” (IDDM) or “juvenile diabetes”; results from the failure of the pancreas to produce enough insulin), Type 2 (also known as “non-insulin-dependent diabetes mellitus” (NIDDM) or “adult-onset diabetes”; results from the failure of cells to respond to insulin properly), and gestational diabetes (seen during pregnancy when high blood sugar levels are observed in the absence of a previous history of diabetes). Many serious complications are observed in diabetic patients including, but not limited to, chronic wounds such as diabetic foot ulcers (also known as diabetic ulcers).

In some embodiments, the subject to be treated by the methods described herein suffers from a severe wound, for example, having an ulcer with an area greater than 2 cm² (e.g., 3 cm², 4 cm² or 5 cm²). In some examples, the subject suffers from one or more plantar ulcers.

Embodiments as described herein can be administered to a subject in one or more doses. Those of skill will readily appreciate that dose levels can vary as a function of the specific the formulation or pharmaceutical composition administered, the severity of the wound, the severity of the symptoms and the susceptibility of the subject to side effects. Advantageous dosages for a given compound are readily determinable by those of skill in the art by a variety of means.

In an embodiment, multiple doses of the formulation can be administered. The frequency of administration of the formulation can vary depending on any of a variety of factors, e.g., the wound, the severity of symptoms, and the like. For example, in an embodiment, the formulation can be administered once per month, twice per month, three times per month, every other week (qow), once per week (qw), twice per week (biw), three times per week (tiw), four times per week, five times per week, six times per week, every other day (qod), daily (qd), twice a day (qid), three times a day (tid), or four times a day. In an embodiment, the formulation or pharmaceutical composition is administered 1 to 4 times a day over a 1 to 10-day time period.

The duration of administration of the formulation, e.g., the period of time over which the formulation is administered, can vary, depending on any of a variety of factors, e.g., patient response, etc.

The amount of the formulations as described herein that can be effective in treating the condition or disease can be determined by standard clinical techniques. In addition, in vitro or in vivo assays can be employed to help identify optimal dosage ranges. The precise dose to be employed can also depend on the route of administration, and can be decided according to the judgment of the practitioner and each patient's circumstances.

Embodiments of the disclosure provide methods and formulation for the administration of the active agent(s) to a subject (e.g., a human) using any available method and route suitable for drug delivery, including in vivo and ex vivo methods, as well as systemic and localized routes of administration. Routes of administration include intranasal, intramuscular, intratracheal, subcutaneous, intradermal, intravitreal, topical application, intravenous, rectal, nasal, oral, and other enteral and parenteral routes of administration. Routes of administration can be combined or adjusted depending upon the agent and/or the target effect. An active agent can be administered in a single dose or in multiple doses.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to affect systemic or local delivery of the composition. For systemic delivery, administration can involve invasive or systemically absorbed topical or mucosal administration of pharmaceutical preparations. In an embodiment, the composition or pharmaceutical composition can also be delivered to the subject by enteral administration. Enteral routes of administration include, but are not limited to, oral and rectal (e.g., using a suppository) delivery.

Methods of administration of the formulation through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission can be accomplished using commercially available “patches” that deliver their product continuously via electric pulses through unbroken skin for periods of several days or more.

In embodiments, the recombinant polypeptide-permeated bandage or wound dressing can be implanting onto a prepared site on or within a subject in need thereof; thereby grafting to a subject the polymer-permeated graft. In other embodiments, the recombinant polypeptide-permeated bandage or wound dressing can be implanted onto a site on or within a subject prior to such site being cleaned and/or prepared, such as in an emergency setting. In such instances, the recombinant serine protease inhibitor polypeptide permeated bandage or wound dressing can prevent subsequent infect, reducing scarring, and/or prepare the site for healing.

### Kits

Aspects of the invention also provides kits comprising recombinant polypeptides or fragments thereof as described herein. Such kits can include one or more containers comprising a recombinant polypeptides or fragments thereof, or a nucleic acid molecule encoding the same, alone or provided as a topical formulation as described herein.

In some embodiments, the kit can comprise instructions for use in accordance with any of the methods described herein. The included instructions can comprise a description of administration of the formulation to promote wound healing according to any of the methods described herein. The kit can further comprise a description of selecting an individual suitable for treatment based on identifying whether that individual has wounds in need of treatment.

The instructions relating to the use of a formulation can include information as to dosage, dosing schedule, and route of administration for the intended treatment. The containers can be unit doses, bulk packages (e.g., multi-dose packages) or sub-unit doses. Instructions supplied in the kits of the invention can be written instructions on a label or package insert (e.g., a paper sheet included in the kit), but machine-readable instructions (e.g., instructions carried on a magnetic or optical storage disk) are also acceptable.

The label or package insert indicates that the composition is used for promoting wound healing. Instructions can be provided for practicing any of the methods described herein.

The kits of this invention are in suitable packaging. Suitable packaging includes, but is not limited to, vials, bottles, jars, flexible packaging (e.g., sealed Mylar or plastic bags), and the like. At least one active agent in the composition is an active agent selected from the group consisting of a recombinant serine protease inhibitor polypeptide and/or a nucleic acid molecule encoding a disclosed recombinant serine protease inhibitor polypeptide.

Kits can optionally provide additional components such as interpretive information. Normally, the kit comprises a container and a label or package insert(s) on or associated with the container. In some embodiments, the invention provides articles of manufacture comprising contents of the kits described herein.

### OTHER EMBODIMENTS

Other compositions, compounds, methods, features, and advantages of the disclosure will be or become apparent to one having ordinary skill in the art upon examination of the following drawings, detailed description, and examples. It is intended that all such additional compositions, compounds, methods, features, and advantages be included within this description, and be within the scope of the disclosure as described herein.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Recombinant Epidermal Serine Protease Inhibitor for Wound Healing and Tissue Repair

Described herein are recombinant serine protease inhibitor (human SerpinB3/B4 or mouse-derived Serpinb3a) for accelerated/improved wound healing and tissue repair by mediating EMT-like processes necessary for wound closure. This recombinant protein therapeutic can be useful for wound types which are characterized by impaired re-epithelialization such as diabetic, infected, and aged wounds, burns and venous stasis ulcers.

### Background

Dermal wound healing relies on a complex, highly regulated orchestration of diverse signaling events. Dysregulation at any stage of healing leads to scarring and tissue disruption and increases morbidity and mortality of associated common comorbidities such as diabetes. The epidermis is the topmost layer of the skin is composed primarily of epithelial cells called keratinocytes. Re-epithelialization is a process in wound healing which activates and mobilizes keratinocytes to migrate into the wound and facilitate tissue closure. In order to migrate into the wound, keratinocytes undergo a process of partial epithelial-to-mesenchymal transition (EMT) involving downregulation of anchorage proteins, upregulation of motility-mediating proteins which resolves (mesenchymal-to-epithelial transition, MTE) as neo-epidermis formation completes. The mechanisms of keratinocyte partial EMT are a topic of investigation. Delayed re-epithelialization is a characteristic of non-healing diabetic wounds and treatments to enhance wound re-epithelialization are likewise under investigation.

SerpinB3 and SerpinB4 belong to the serine protease inhibitor (serpin) superfamily or proteins. SerpinB3 and SerpinB4 are Great Ape-lineage duplications of the mouse ortholog Serpinb3a. At high levels of expression, functional SerpinB3 can be released from cells with no effect on cell viability and studies on paracrine function by exogenous treatment of cells with recombinant SerpinB3 demonstrated the induction of EMT-like states in cultured cells (Quarta et al., J Pathol 2010). Gene expression of SerpinB3 and SerpinB4 in wounds from elderly subjects are approximately 10- to 20-fold lower than young subjects (Hardman and Ashcroft, Genome Bio 2008) and protein levels of SerpinB3 were found to be reduced in wound tissue from non-healing diabetic wounds versus rapidly healing diabetic wounds (Fadini et al., Diabetologia 2014).

### Non-Limiting, Exemplary Data

Dataset mining (NCBI GEO GSE23006; Chen et al., BMC Genomics 2010) indicated a time-dependent induction of Serpinb3a expression in mouse dermal wounds, but not mucosal wounds, indicating a skin-specific role of Serpinb3a (human SerpinB3/B4) in wound repair. This time-dependent upregulation and resolution of Serpinb3a in dermal wounds was supported experimentally in Balb/c mice. Further dataset mining (NCBI GEO GSE141956) indicated that migrating keratinocytes in diabetic mouse wounds have significantly lower expression of Serpinb3a than migrating keratinocytes in wounds of healthy mice. Together, these results indicate a temporal role for Serpinb3a (or human SerpinB3 and SerpinB4) in wound healing which can be expressed in healthy healing and which is impaired in poor healing.

Herein we describe recombinant protein Serpinb3a (mouse-derived), constituting the function of both human SerpinB3 and human SerpinB4, or consensus mutants containing relevant substitutions from the human SerpinB3 and/or SerpinB4, or a reactive center loop truncation mutants of Serpinb3a, SerpinB3 or SerpinB4 or of consensus mutants containing relevant substitutions into Serpinb3a from human SerpinB3 or SerpinB4 in addition to reactive center loop truncation.

Mouse Serpinb3a (NCBI Accession NP_033152.3):
SEQ ID NO: 1)
MHLFAEATTKFTLELYRQLRESDNNIFYSPISMMTALAMLQLGAKGNTEK
QIEKVLQFNETTKKTTEKSAHCHDEENVHEQFQKLMTQLNKSNDAYDLKA
ANSIYGAKGFPFVQTFLEDIKEYYQANVESLDFEHAAEESEKKINSWVES
QTNGKIKDLFPNGSLNRSTIMVLVNAVYFKGQWNHKFDEKHTTEEKFWLN
KNTSKPVQMMKQNIEFNFMFLEDVQAKIVEIPYKGKELSMIVLLPVEING
LKQLEEQLTADKLLEWTRAENMHMTELYLSLPRFKVDEKYDLPIPLEHMG
MVDAFDPQKADFSGMSSTQGLVVSKVLHKSFVEVNEEGTEAAAATGVEVS
LTSAQIAEDFCCDHPFLFFIIHRKTNSILFFGRISSP
Human SerpinB3 (NCBI Accession NP_008850.1):
(SEQ ID NO: 2)
MNSLSEANTKFMFDLFQQFRKSKENNIFYSPISITSALGMVLLGAKDNTA
QQIKKVLHFDQVTENTTGKAATYHVDRSGNVHHQFQKLLTEFNKSTDAYE
LKIANKLFGEKTYLFLQEYLDAIKKFYQTSVESVDFANAPEESRKKINSW
VESQTNEKIKNLIPEGNIGSNTTLVLVNAIYFKGQWEKKFNKEDTKEEKF
WPNKNTYKSIQMMRQYTSFHFASLEDVQAKVLEIPYKGKDLSMIVLLPNE
IDGLQKLEEKLTAEKLMEWTSLQNMRETRVDLHLPRFKVEESYDLKDTLR
TMGMVDIFNGDADLSGMTGSRGLVLSGVLHKAFVEVTEEGAEAAAATAVV
GFGSSPTSTNEEFHCNHPFLFFIRONKTNSILFYGRFSSP
Human SerpinB4 Isoform 1 (NCBI Accession
NP_002965.1):
(SEQ ID NO: 3)
MNSLSEANTKFMFDLFQQFRKSKENNIFYSPISITSALGMVLLGAKDNTA
QQISKVLHFDQVTENTTEKAATYHVDRSGNVHHQFQKLLTEFNKSTDAYE
LKIANKLFGEKTYQFLQEYLDAIKKFYQTSVESTDFANAPEESRKKINSW
VESQTNEKIKNLFPDGTIGNDTTLVLVNAIYFKGQWENKFKKENTKEEKF
WPNKNTYKSVQMMRQYNSFNFALLEDVQAKVLEIPYKGKDLSMIVLLPNE
IDGLQKLEEKLTAEKLMEWTSLQNMRETCVDLHLPRFKMEESYDLKDTLR
TMGMVNIFNGDADLSGMTWSHGLSVSKVLHKAFVEVTEEGVEAAAATAVV
VVELSSPSTNEEFCCNHPFLFFIRQNKTNSILFYGRFSSP

In embodiments, there are the 20 amino acids constituting the reactive center loop (RCL) from the hinge region through the variable loop containing the P1/P1′ scissile bond (indicated in brackets):

Serpinb3a:
(SEQ ID NO: 8)
G (338) TEAAAATGVEVSL [TS] AQIA
SerpinB3:
(SEQ ID NO: 9)
G (340) AEAAAATAVVGFGS [SP] TST
SerpinB4:
(SEQ ID NO: 10)
G (340) VEAAAATAVVVVEL [SS] PST

Reactive center loop truncation mutants will contain an early stop codon immediately after the numbered Glycine (G) indicated in the sequences herein.

Protein is produced by cloning the full coding sequence (or RCL truncation mutant) into the pET28a(+) bacterial expression vector to introduce an N-terminal polyhistidine tag driven by an IPTG-inducible T7 promotor. Expression can be performed in BL21 (DE3) E. coli or in ClearColi LPS-deficient E. coli. Protein is purified by Nickel-NTA affinity purification followed by secondary purification by anion exchange (e.g., by Mono-Q column). His-tag can be removed by incorporation of a TEV protease consensus sequence EXLYΦQ\φ (SEQ ID NO: 21) where X is any residue, Φ is any large/medium hydrophobic residue and φ is any small hydrophobic or polar residue. Alternatively, polyhistidine tag-free purification can be performed by expression in bacteria followed by fast protein liquid chromatography (FPLC). Confirmation of protein purity is performed by SDS-PAGE followed by Coomassie or silver staining. Protease inhibitory function is confirmed by residual activity assay against cathepsin L (cysteine protease; Serpinb3a or SerpinB3) or cathepsin G (serine protease; Serpinb3a or SerpinB4).

In embodiments, protein can be delivered to a wound topically in saline solution, or in a topical formulation comprising one or more carriers and excipients, including viscosity increasing agents, ointment bases, antimicrobial preservatives, temperature and pH sensing probes, emulsifying agents and/or solvents.

In embodiments, the recombinant Serpinb3a/B3/B4/truncation protein can be provided in a sustained-release vehicle, hydrogel, or dressing which can be composed of a variety of organic polymers (natural or synthetic), including but not limited to chitosan, alginate, polyphosphazenes, polyacrylates and similar. These compositions can be further modified by inclusion of biologic crosslinkers such as collagen, fibrinogen in the presence of thrombin and similar.

Therapeutic doses can range from 1 ng/kg bodyweight to 1 mg/kg bodyweight and can be given daily, every other day, or as otherwise indicated by specific wound management needs.

### Example 2—Recombinant Epidermal Serine Protease Inhibitor for Wound Healing and Tissue Repair

Dermal wound healing is a complex and highly regulated process. Certain wounds are characterized by impaired re-epithelialization which prevents wound closure. Examples include diabetic wounds, infected wounds, aged wounds, burns and venous stasis ulcers. There are no existing treatments that specifically target re-epithelialization for wound healing treatment. In embodiments, the technology described herein is directed to chronic wound healing via re-epithelialization using a serine protease inhibitor.

Recombinant serine protease inhibitor (Human Serpin B3/B4 or mouse-derived Serpinb3A used) produced in E. coli and modified to have no glycosylation (common when produced in E. coli), C-terminus modification to help with purification and codon optimized for E. coli production.
Chimera recombinant SerpinB3 with mouse and human can be produced
Developing GMP production of recombinant protein
Time-dependent induction of Serpinb3a expression in mouse dermal wounds but not mucosal wounds (indicates skin specificity)
Therapeutic doses 1 ng/kg to 1 mg/kg bodyweight, daily or longer
A non-limiting exemplary embodiment of delivery: time released in hydrogel or wound dressing (natural or synthetic, e.g. chitosan, alginate, polyphosphazenes, polyacrylates).
Can also include biologic crosslinkers-collagen, fibrinogen with thrombin

### Example 3—Role of Epidermal Serpins in Wound Healing

### Abstract

Fine-tuned regulation of the complex signaling initiated by cutaneous wounding is essential for appropriate tissue repair [1-3]. Dysregulation at any stage of healing leads to scarring and tissue disruption and contributes to the increased morbidity and mortality associated with common comorbidities such as diabetes. Re-epithelialization is a process in wound healing now understood to involve a partial, transient epithelial-to-mesenchymal transition (EMT)-like state which allows keratinocytes in the epidermis to mobilize towards the wound bed [4]. The precise mechanisms regulating transient epidermal EMT in wound repair remain unknown. SerpinB3/B4 (mouse Serpinb3a) are members of the serine protease inhibitor (serpin) superfamily and were originally identified as potent autocrine and paracrine drivers of EMT in cancer [5]. Evidence indicates that SerpinB3/B4 are expressed in keratinocytes and induced upon cutaneous wounding. Protein levels of wound bed SerpinB3/B4 have been indicated as a healing biomarker for diabetic wounds, but a specific mechanistic role of SerpinB3/B4 in wound repair has not yet been described [6]. Without wishing to be bound by theory, we can describe that transient epidermal EMT and re-epithelialization during wound repair is mediated by SerpinB3/B4. In addition to signaling mechanisms, we will further investigate therapeutic delivery of recombinant SerpinB3/B4 to alleviate the delayed epidermal EMT exhibited by diabetic wounds. Without wishing to be bound by theory, these studies will provide a unique wound repair mechanism and explore a new therapeutic target.

### Non-Limiting, Exemplary Aims

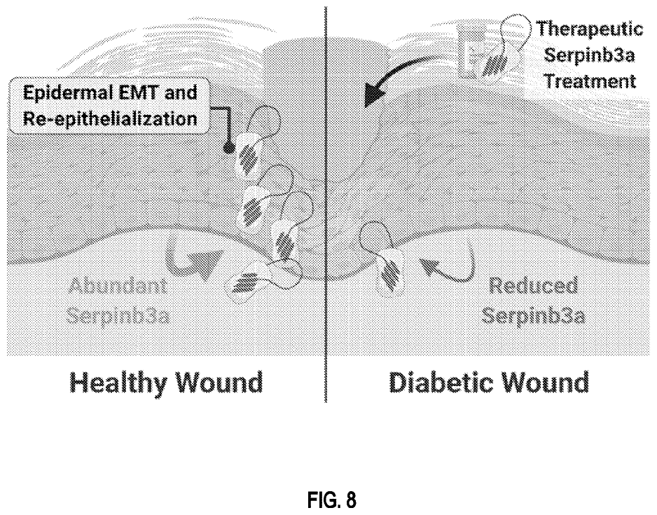

In embodiments, in vivo models, paired with histopathologic and biochemical analyses can elucidate the role of SerpinB3/B4 (mouse Serpinb3a) in acute and diabetic cutaneous wound healing. Further, the role of recombinant SerpinB3/B4 to enhance diabetic wound healing can be explored. (FIG. 8)

Study 1: Define the role of SerpinB3/B4 (Serpinb3a) in acute and diabetic wound healing. Study 2: Determine the effect of recombinant SerpinB3/B4 (Serpinb3a) in diabetic wounds.

No mechanistic studies have investigated the role of SerpinB3/B4 in wound repair. These studies are based on the inclusion of SerpinB3/B4 in wound healing and identification of low levels being associated with diabetic wound impairment, the role for SerpinB3/B4 in EMT processes, and the ability to therapeutically deliver recombinant serpins to wounds. Together, these indicate that SerpinB3/B4 can play a critical role and be a therapeutic target in the wound repair process. Without wishing to be bound by theory, these studies will have a transformative impact on wound healing by elucidating a new, druggable mechanistic pathway involved in acute and diabetic wound repair with a strong path to clinical translation.

Dermal wounds are a medical burden. More than 6 million chronic cutaneous wound cases amount to a cost of over $20 billion per year in health management costs in the USA, equating to approximately 5% of the total cost of Medicare and Medicaid [10]. Tissue repair in the skin proceeds along continuous and overlapping phases of (i) hemostasis, (ii) inflammation, (iii) proliferation and (iv) remodeling [1-3]. Dysregulation in the onset or resolution of any stage leads to impaired healing and complex comorbidities such as diabetes commonly result in impaired wound repair. Diabetic patients have a 15% lifetime risk or chronic foot ulcers, which remain the primary cause for amputation and result in significant negative emotional, physical and financial costs [11]. Despite intense investigation into new advanced treatments such as growth factors, acellular matrices and stem cell therapies, a better understanding of the molecular and cellular differences between healing and nonhealing wounds is needed to develop more effective therapeutic approaches [12].

Delayed re-epithelialization is a well-recognized characteristic of non-healing diabetic wounds and treatments to specifically enhance wound re-epithelialization by activating and mobilizing keratinocytes are under investigation [13,14]. The partial EMT-like state exhibited by keratinocytes is a critical, transient phenotypic switch during re-epithelialization [4]. While the process involves some of the same signaling partners (transcription factors, junctional proteins, etc.) in re-epithelialization as it does in cancer, its role in healing remains incompletely characterized and it has been indicated that understanding the differences and similarities between EMT in cancer versus re-epithelialization will reveal insights leading to improvements in wound care [15,16]. Without wishing to be bound by theory, these studies will elucidate a new mechanism of regulation for epidermal EMT in wound healing mediated by SerpinB3/B4, a protein canonically associated with cancer EMT.

Herein we describe a new autocrine/paracrine pathway to expand the fundamental understanding of re-epithelialization in healthy and diabetic wound healing, and validate new therapeutic treatments for wound repair by complementation with a recombinant version of the endogenously expressed EMT mediator.

### Background

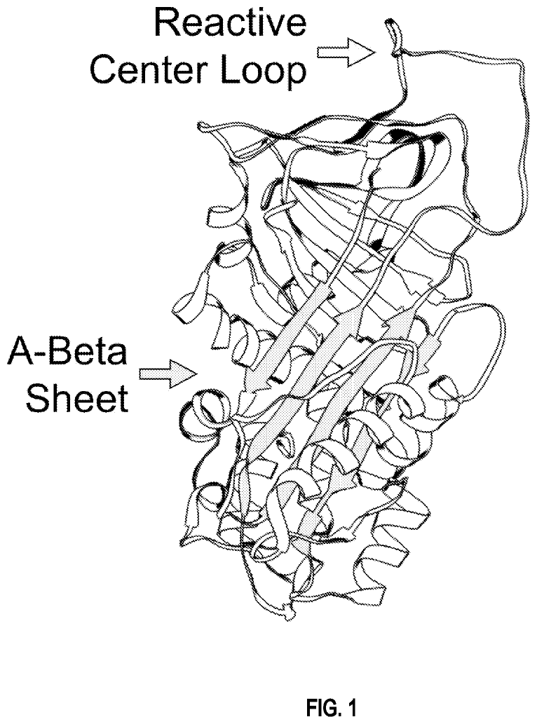

The serine protease inhibitors (serpins) are an ancient and diverse protein superfamily found in all branches of the Tree of Life [17]. Serpins are expressed in all tissues of the body and are involved in the regulation of a wide array of physiologic processes [18]. Serpins are characterized by a metastable structure with two components: a reactive center loop (RCL) and a 4-stranded core beta-sheet (the “A” beta-sheet) (FIG. 1) [19]. The RCL contains a protease recognition sequence which acts as a bait for the target activated protease. Cleavage of the RCL triggers a dramatic rearrangement wherein the RCL “swings” 70 angstroms across the protein and inserts itself as the third strand in a now 5-stranded beta-sheet before the enzyme-substrate Michaelis complex can separate resulting in a covalently bonded “suicide complex.” This rearrangement permanently disables both the protease and serpin which are then removed by intra- and extracellular degradation. Serpins can also signal non-canonically by stimulating cellular responses independent of protease inhibitor activity through poorly understood mechanisms [20]. The modularity of the serpin structure allows evolutionary diversification by small changes in the RCL. For example, human SerpinB3 and SerpinB4 (originally Squamous Cell Carcinoma Antigens [SCCA]-1 and -2, respectively) are early Great Ape duplications of mouse Serpinb3a. While SerpinB3 exhibits exclusively cysteine protease inhibitor activity and SerpinB4 is exclusively a serine protease inhibitor, pre-duplication mouse Serpinb3a is a cross-class cysteine and serine protease inhibitor [21].

Dysregulated expression SerpinB3/B4 have been associated with numerous cancers and chronic fibrotic diseases [5]. Overexpression studies of SerpinB3/B4 demonstrate effects on TGF-beta modulation and stimulation of EMT via the unfolded protein response and IL-6 signaling in cancers and chronic liver disease [22,23]. Despite belonging to the Clade B ovalbumin-like intracellular serpins, sufficient expression results in extracellular release of functional SerpinB3 with no negative effect on cell viability [24]. This extracellular released SerpinB3 can act in an autocrine and paracrine manner and treatment of non-expressing epithelial cancer cells with recombinant, exogenous SerpinB3 can stimulate EMT as indicated by a loss of E-cadherin, an increase in vimentin and a nuclear localization of beta-catenin [24]. Thus, exposure to functional SerpinB3/B4 is sufficient to stimulate EMT-like processes.

In mice, Serpinb3a is expressed in tissues, including epidermal keratinocytes [25]. In a pathologic sense, higher Serpinb3a was found to contribute to epidermal barrier dysfunction in a mouse model of atopic dermatitis [26]. Transcriptome studies of human skin graft donors identified SerpinB4 among the most upregulate genes in wound boundary following superficial wounding [27]. Proteomic studies of human burn wounds identified a significant upregulation of SerpinB3/B4 [28]. SerpinB3 was found by bulk proteomics to be enriched in healing versus nonhealing wounds in diabetic patients, acting as a healing biomarker with 75% sensitivity and 62.5% specificity [6]. In this same study, human SerpinB3 was overexpressed under the Alpha-1-antitrypsin promoter in mice and found an improvement in STZ-induced diabetic mice with wounds complicated by hindlimb ischemia. However, few if any conclusions can be drawn regarding the role of SerpinB3/B4 or Serpinb3a from these studies because (1) endogenous Serpinb3a function or dynamics was not investigated: (2) SerpinB3 does not have the totality of function of mouse Serpinb3a [21], and (3) promoter mismatch can be responsible for artifact, as SerpinB3/B4 is known to be regulated by the wound healing-associated transcription factor STAT3 and expression is low in healthy skin [30], but was high in healthy skin in the study. Without wishing to be bound by theory, SerpinB3/B4 have a role of healthy and diabetic wound repair by an as-yet unknown mechanism.

### Non-Limiting, Exemplary Data

Transient Serpinb3a expression is induced after injury, for example, in the skin. Affymetrix Mouse Genome 430 2.0 Array data was queried using the GEO2R tool in the NCBI Gene Expression Omnibus (GEO) repository (Dataset GSE23006) [31]. 1-mm punch biopsies were used to produce full-thickness wounds on the dorsum or tongue of 8-week old Balb/c mice and tissue was collected at the indicated timepoint using a 2-mm biopsy punch [31].

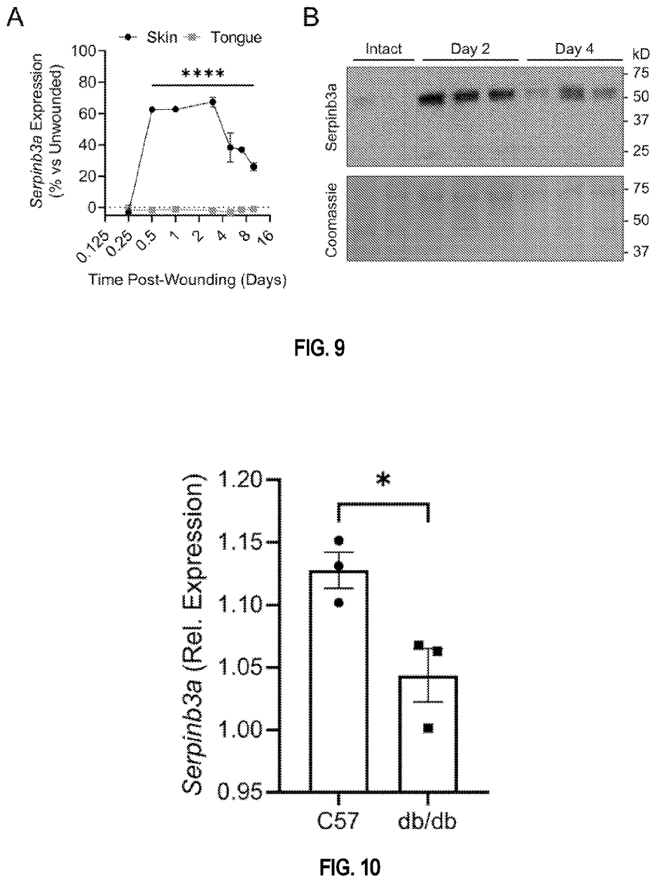

Herein, post-analysis was performed and skin-specific Serpinb3a gene expression upregulation after injury was identified, which was not observed during mucosal healing in the tongue (FIG. 9 panel A). This is corroborated by immunoblot analysis of peri-wound skin tissues collected from 8-week old Balb/c mice with 5-mm biopsy punch full-thickness wounds which demonstrate a peak of Serpinb3a protein at Day 2 which begins to resolve by Day 4 post-wounding (FIG. 9 panel B).

Migrating wound-edge keratinocytes of diabetic mice have reduced Serpinb3a expression. Affymetrix Mouse Gene 1.0 ST Array data was queried using the GEO2R tool in the NCBI GEO repository (Dataset GSE141956). In this dataset, wounds were harvested at 3-days post-wounding from C57BL6/J and db/db mice and migrating keratinocytes identified by E-cadherin IHC staining were isolated by laser capture microdissection and expression analyzed. Post-analysis indicates that migrating keratinocytes from db/db mice have significantly lower relative expression of Serpinb3a than in migrating keratinocytes from wildtype mice (FIG. 10).

### Non-Limiting, Exemplary Experimental Approach

Study 1: Define the role of SerpinB3/B4 (Serpinb3a) in acute and diabetic wound healing.

Without wishing to be bound by theory, transient epidermal EMT and re-epithelialization during wound repair is positively regulated by SerpinB3/B4 (Serpinb3a).

1A. Validation of the role of Serpinb3a in acute wound healing. Wound healing will be studied in vivo in 8-week old wildtype Balb/c and Serpinb3a−/− mice (C.129-Serpinb3 atm1Gsil/J on a Balb/c background, JAX catalog #017740). Full thickness excisional wounds (5 mm diameter, approximately 0.5 mm depth, preserving the panniculus carnosus) will be made using biopsy punch and prevented from contraction by affixing a Tegaderm-covered silicone splint with sutures to force healing by re-epithelialization (as in humans) as previously described [34]. Mice will be followed for up to 21 days. Early follow-up for tissue collection will be performed on days 2, 4, 7 and 10 post-wounding, which can overlap the full range of Serpinb3a expression based on data. Wound planimetry will be performed by measuring calibrated images of wounds collected daily. On each day of the study, the wound region will be evaluated for barrier function using trans epidermal water loss (TEWL, a non-destructive test) with a vapometer and compared to a distal, intact skin region on the same mouse.

Histopathologic and biochemical analyses: At the indicated follow-up times, wound tissues will be collected (3 cm square tissue segment including the wound site) and fixed in 10% neutral-buffered formalin for 24 hours, transferred to ethanol for storage and analyzed for histopathology, or snap-frozen and stored at −80° C. for biochemical analysis. Tissues will be embedded in paraffin and 4-6 μm sections will be stained. Hematoxylin and eosin (H&E) staining will be examined for dermal gap measurements and Masson's Trichrome (MT) will be assessed for collagen content [37]. A healing score will be performed by a blinded, board-certified dermatopathologist for each animal based on (1) bridging of the incision site, (2) degree of inflammation by H&E staining. Bridging will be based on tissue continuity, re-epithelialization and granulation tissue formation, with each given a score of none (1), poor (2), moderate (3), good (4), or excellent and complete (5). The dermal gap measurements (i.e., the distance between leading edges of the wounds) will be quantified based on literature methods [36]. Fibrosis (i.e., collagen deposition) will be scored [37] on MT-stained tissue sections with each given a score of absent-to-mild (1), moderate (2) or extensive (3). Immunohistochemistry (IHC) will be performed for Serpinb3a and for EMT markers: E-cadherin, Vimentin, Zeb1, Slug, and β-catenin; and healing markers: proliferation (Ki67), keratinocytes (cytokeratin 5 and 10), myofibroblasts (alpha-smooth muscle actin), vascularization (CD31) and scarring (collagen III and I). Automated, unbiased image analysis algorithms will be performed using the ImageJ/FIJI software to quantify DAB positivity [34,38]. Immunoblot and/or ELISA (depending on availability) will be performed for Serpinb3a and the EMT markers listed herein.

1B. Determination of the dynamics of Serpinb3a in diabetic wound healing. Wound healing will be studied in 12-week old db/db mice (BKS.Cg-Dock7m+/+ Leprdb/J, JAX catalog #000642) and wildtype controls from the colony provided by the Jackson Laboratory. Full-thickness wounds will be performed as described herein. Mice will be followed for up to 21 days. Early follow-up for tissue collection will be performed on days 2, 4, 7, 10 and 14 post-wounding, with the extra 14-day time point to account for a delay in expression dynamics due to the diabetic state. Wound planimetry and barrier function will be performed as described herein. Tissue will be collected at the indicated timepoints and histological and biochemical analysis with H&E, MT, ELISA and immunoblot will be performed as described herein.

Non-limiting, Exemplary Results and Strategies. Without wishing to be bound by theory, Serpinb3a−/− mice will exhibit a delay in wound repair due to impaired epidermal EMT and re-epithelialization. Further, Serpinb3a will exhibit (i) delayed or (ii) reduced expression in diabetic wounds. The studies are designed to provide sufficient resolution to cover the full timeline of the EMT-like state observed in the wounded epidermis with a comprehensive histologic and biochemical workup which will ensure a full evaluation of epidermal dynamics with and without expression of Serpinb3a. Utilizing an in vivo system reduces any artifacts which can be observed in monolayer scratch assays. The selection of db/db mice is based on the common use of this strain in the wound healing literature. However, an alternative strain, NONcNZO10, will soon be available and exhibit multigenic diabetes with wound healing impairment described to be more similar to human clinical features and are already planned in NIH-funded studies in the lab. When these mice are available and studies begin, the planned histological and biochemical analyses for Serpinb3a and EMT markers will also be performed on these tissues.

Aim 2: Determine the effect of recombinant SerpinB3/B4 (Serpinba) delivery in diabetic wounds.

Without wishing to be bound by theory, recombinant Serpinb3a will improve diabetic wound healing by rescuing impaired epidermal EMT and re-epithelialization.

2A. Generation of recombinant and mutant inactive Serpinb3a. Recombinant wildtype Serpinb3a and a C-terminal mutant with the complete RCL deleted, beginning at the hinge region (G338; Serpinb3aΔRCL) will be generated as previously reported [24]. Briefly the complete coding regions will be synthesized by a commercial source and inserted into the pET28a bacterial expression vector between the XbaI and XhoI restriction sites, maintain inclusion of a C-terminal 6×-Histag (SEQ ID NO: 17). Purified plasmid DNA will be transformed into ClearColi BL21 (DE3) chemically competent bacteria for endotoxin-free expression of recombinant protein by IPTG induction with purification by Ni-NTA column [40]. Protein purity in collected fractions will be evaluated by SDS-PAGE with Coomassie staining and considered pure at a level ≥95%. Endotoxin levels will be confirmed by FDA-approved LAL assay and maintained within the limit 5 endotoxin units (EU)/kg, or 0.1 EU/20g mouse according to the FDA draft guidance FDA-2020-D-1294. Functional activity of the produced protein will be tested against commercially available recombinant Cathepsin L (CatL, cysteine protease) and Cathepsin G (CatG, serine protease) by measuring the ability for the recombinant serpins to prevent the cleavage of the fluorogenic substrate Z-LR-AMC (CatL) and chromogenic N-Succinyl-AAPF-pNA (SEQ ID NO: 22) (CatG) as previously described [41].

2B. Evaluation of the therapeutic utility of Serpinb3a in diabetic wound healing. Wound healing will be validated in 12-week old db/db mice and wildtype controls as describe in section 1B. Mice will be separated into low dose (100 ng/g) and high dose (1 μg/g) groups for each treatment, Serpinb3a and Serpinb3aΔRCL, and a saline control group for each strain (saline controls are necessary in this study as isoflurane is required for application of treatment through the silicon splint). These doses were selected based on prior experience using a dose of 100 ng/g of therapeutic serpin in a wound healing model [7]. This dose is considered unusually low for therapeutic biologics (μg/kg range) and thus a 10× dose is also planned (mg/kg range). Mice will be treated by topical application of recombinant protein in normal saline solution on the day of wounding, and on days 3 and 5 post-wounding as previously described [7,8]. Initially, mice will be allowed to heal for 21 days. If wound healing is improved by treatment with one of the dose groups of Serpinb3a, the best performing dose group will be repeated for intermediate follow-up at days 3, 7 and 14 post-wounding for intermediate day characterization. Wound planimetry and barrier function will be performed as described in section 1A. Tissue will be collected at the indicated timepoints and histological and biochemical analysis with H&E, MT, ELISA and immunoblot will be performed as described in section 1A.

Non-limiting exemplary results: Without wishing to be bound by theory, Serpinb3a and Serpinb3aΔRCL will be successfully produced and characterized as performed in procedures for other serpins [19]. Without wishing to be bound by theory, the application of recombinant Serpinb3a, but not Serpinb3aΔRCL, will improve wound healing in diabetic mice based on the published evidence discussed herein that (i) low levels of human SerpinB3 are diagnostic for nonhealing diabetic wounds, (ii) a delayed switch to an EMT-like state is characteristic for diabetic wounds, and (iii) treatment with exogenous recombinant SerpinB3 induced EMT in vitro. Vertebrate Animal Use and Data Analysis

Based on prior work [34] and preliminary results, considering an effect size of between 1.5 and 2 (Cohen's d) and a prior power analysis using a two-tailed test at effect size of 1.5, a error probability of 0.05, Power of 0.8 and an allocation ratio of 1, 6 mice/group will be sufficient for statistically significant closure. A 15% overage for incidentals are planned. Mice groups are planned as follows: Section 1A-Balb/c (6*5 time points*1.15-35 mice), Serpinb3a−/− (6*5 time points*1.15=35 mice); Section 1B—dbdb (6*6 time points*1.15=42 mice), wildtype controls (6*6 time points*1.15=42 mice); Section 2B—Initial—dbdb (6*5 treatment groups*1 time point*1.15=35 mice), wildtype controls (6*5 treatment groups*1 time point*1.15=35 mice). Section 2B—Follow-up—dbdb (6*2 groups {treatment and saline}*3 time points*1.15=42 mice), wildtype controls (6*2 groups {treatment and saline}*3 time points*1.15=42 mice). In total, 35 Balb/c, 35 Serpinb3a−/−, 77 dbdb and 77 matched wildtype controls are planned with a potential for an additional 42 dbdb (total 119 dbdb) and 42 matched wildtype controls (total 119 wildtype controls) are planned for these studies. Quantitative differences among groups will be compared using one-way ANOVA with multiple comparisons analysis using Tukey post-hoc or Kruskal Wallis H tests. Statistics will be performed with Graphpad Prism using two-sided tests and significant p-value threshold at <0.05.

We will investigate the molecular signaling mechanisms of Serpinb3a and the ability to develop a hydrogel-based Serpinb3a formulation.

### References Cited in this Example

1. Singer, A. J.; Clark, R. A. F. Cutaneous Wound Healing. N. Engl. J. Med. 1999, 341, 738-746.
2. Sun, B. K.; Siprashvili, Z.; Khavari, P. A. Advances in skin grafting and treatment of cutaneous wounds. Science (80-.). 2014, 346, 941-945.
3. Reinke, J. M.; Sorg, H. Wound Repair and Regeneration. Eur. Surg. Res. 2012, 49, 35-43.
4. Haensel, D.; Dai, X. Epithelial-to-mesenchymal transition in cutaneous wound healing; Where we are and where we are heading. Dev. Dyn. 2018, 247, 473-480.
5. Sun, Y.; Sheshadri, N.; Zong, W. X. SERPINB3 and B4; From biochemistry to biology. Semin. Cell Dev. Biol. 2017, 62, 170-177.
6. Fadini, G. P.; Albiero, M.; Millioni, R.; Poncina, N.; Rigato, M.; Scotton, R.; Boscari, F.; Brocco, E.; Arrigoni, G.; Villano, G.; et al. The molecular signature of impaired diabetic wound healing identifies serpinB3 as a healing biomarker. Diabetologia 2014, 57, 1947-1956.
7 Zhang, L.; Yaron, J. R.; Tafoya, A. M.; Wallace, S. E.; Kilbourne, J.; Haydel, S.; Rege, K.; McFadden, G.; Lucas, A. R. A Virus-Derived Immune Modulating Serpin Accelerates Wound Closure with Improved Collagen Remodeling. J. Clin. Med. 2019, 8, 1626.
8. Yaron, J. R.; Zhang, L.; Guo, Q.; Awo, E. A.; Burgin, M.; Schutz, L. N.; Zhang, N.; Kilbourne, J.; Daggett-vondras, J.; Lowe, K. M.; et al. Recombinant myxoma virus-derived immune modulator m-t7 accelerates cutaneous wound healing and improves tissue remodeling. Pharmaceutics 2020, 12.
9. Zhang, L.; Yaron, J. R.; Guo, Q.; Kilbourne, J.; Awo, E. A.; Burgin, M.; Schutz, L. N.; Wallace, S. E.; Lowe, K. M.; Lucas, A. R. Topical Application of Virus-Derived Immunomodulating Proteins and Peptides to Promote Wound Healing in Mouse Models; 2021; Vol. 2225;.
10. Niu, Y.; Li, Q.; Ding, Y.; Dong, L.; Wang, C. Engineered delivery strategies for enhanced control of growth factor activities in wound healing. Adv. Drug Deliv. Rev. 2019, 146, 190-208.
11. Singh, N. Preventing Foot Ulcers in Patients With Diabetes. JAMA 2005, 293, 217.
12. Frykberg, R. G.; Banks, J. Challenges in the Treatment of Chronic Wounds. Adv. Wound Care 2015, 4, 560-582.
13. Xiao, Y.; Reis, L. A.; Feric, N.; Knee, E. J.; Gu, J.; Cao, S.; Laschinger, C.; Londono, C.; Antolovich, J.; McGuigan, A. P.; et al. Diabetic wound regeneration using peptide-modified hydrogels to target re-epithelialization. Proc. Natl. Acad. Sci. U.S.A 2016, 113, E5792-E5801.
14. Rousselle, P.; Braye, F.; Dayan, G. Re-epithelialization of adult skin wounds; Cellular mechanisms and therapeutic strategies. Adv. Drug Deliv. Rev. 2019, 146, 344-365.
15. Nieto, M. A.; Huang, R. Y. Y. J.; Jackson, R. A. A.; Thiery, J. P. P. Emt; 2016. Cell 2016, 166, 21-45.
16. Leopold, P. L.; Vincent, J.; Wang, H. A comparison of epithelial-to-mesenchymal transition and re-epithelialization. Semin. Cancer Biol. 2012, 22, 471-483.
17. Yaron, J. R.; Zhang, L.; Guo, Q.; Burgin, M.; Schutz, L. N.; Awo, E.; Wise, L.; Krause, K. L.; Ildefonso,
C. J.; Kwiecien, J. M.; et al. Deriving Immune Modulating Drugs from Viruses—A New Class of Biologics. J. Clin. Med. 2020, 9, 972.
18. Silverman, G. A.; Bird, P. I.; Carrell, R. W.; Church, F. C.; Coughlin, P. B.; Gettins, P. G. W.; Irving, J. A.; Lomas, D. A.; Luke, C. J.; Moyer, R. W.; et al. The serpins are an expanding superfamily of structurally similar but functionally diverse proteins. Evolution, mechanism of inhibition, novel functions, and a revised nomenclature. J. Biol. Chem. 2001.
19. Mahon, B. P.; Ambadapadi, S.; Yaron, J. R.; Lomelino, C. L.; Pinard, M. A.; Keinan, S.; Kurnikov, I.; Macaulay, C.; Zhang, L.; Reeves, W.; et al. Crystal Structure of Cleaved Serp-1, a Myxomavirus-Derived Immune Modulating Serpin; Structural Design of Serpin Reactive Center Loop Peptides with Improved Therapeutic Function. Biochemistry 2018, 57, 1096-1107.
20. Ehlers, M. R. Immune-modulating effects of alpha-1 antitrypsin. Biol. Chem. 2014, 395, 1187-1193.
21. Al-Khunaizi, M.; Luke, C. J.; Askew, Y. S.; Pak, S. C.; Askew, D. J.; Cataltepe, S.; Miller, D.; Mills, D. R.; Tsu, C.; Brömme, D.; et al. The serpin SQN-5 is a dual mechanistic-class inhibitor of serine and cysteine proteinases. Biochemistry 2002, 41, 3189-3199.
22. Turato, C.; Calabrese, F.; Biasiolo, A.; Quarta, S.; Ruvoletto, M.; Tono, N.; Paccagnella, D.; Fassina, G.; Merkel, C.; Harrison, T. J.; et al. SERPINB3 modulates TGF-B expression in chronic liver disease. Lab. Investig. 2010, 90, 1016-1023.
23. Sheshadri, N.; Catanzaro, J. M.; Bott, A. J.; Sun, Y.; Ullman, E.; Chen, E. I.; Pan, J. A.; Wu, S.; Crawford, H. C.; Zhang, J.; et al. SCCA1/SERPINB3 promotes oncogenesis and epithelial-mesenchymal transition via the unfolded protein response and IL6 signaling. Cancer Res. 2014, 74, 6318-6329.
24. Quarta, S.; Vidalino, L.; Turato, C.; Ruvoletto, M.; Calabrese, F.; Valente, M.; Cannito, S.; Fassina, G.; Parola, M.; Gatta, A.; et al. SERPINB3 induces epithelial—Mesenchymal transition. J. Pathol. 2010, 221, 343-356.
25. Sakata, Y.; Arima, K.; Takeshita, K.; Takai, T.; Aoki, S.; Ogawa, H.; Sugihara, H.; Fujimoto, K.; Izuhara, K. Characterization of novel squamous cell carcinoma antigen-related molecules in mice. Biochem. Biophys. Res. Commun. 2004, 324, 1340-1345.
26. Sivaprasad, U.; Kinker, K. G.; Ericksen, M. B.; Lindsey, M.; Gibson, A. M.; Bass, S. A.; Hershey, N. S.; Deng, J.; Medvedovic, M.; Khurana Hershey, G. K. SERPINB3/B4 Contributes to Early Inflammation and Barrier Dysfunction in an Experimental Murine Model of Atopic Dermatitis. J. Invest. Dermatol. 2015, 135, 160-169.
27. Nuutila, K.; Siltanen, A.; Peura, M.; Bizik, J.; Kaartinen, I.; Kuokkanen, H.; Nieminen, T.; Harjula, A.; Aarnio, P.; Vuola, J.; et al. Human skin transcriptome during superficial cutaneous wound healing. Wound Repair Regen. 2012, 20, 830-839.
28. Pollins, A. C.; Friedman, D. B.; Nanney, L. B. Proteomic Investigation of Human Burn Wounds by 2D-Difference Gel Electrophoresis and Mass Spectrometry. J. Surg. Res. 2007, 142, 143-152.
29. Dauer, D. J.; Ferraro, B.; Song, L.; Yu, B.; Mora, L.; Buettner, R.; Enkemann, S.; Jove, R.; Haura, E. B. Stat3 regulates genes common to both wound healing and cancer. Oncogene 2005, 24, 3397-3408.
30. Katagiri, C.; Iida, T.; Nakanishi, J.; Ozawa, M.; Aiba, S.; Hibino, T. Up-regulation of serpin SCCA1 is associated with epidermal barrier disruption. J. Dermatol. Sci. 2010, 57, 95-101.
31. Chen, L.; Arbieva, Z. H.; Guo, S.; Marucha, P. T.; Mustoe, T. A.; DiPietro, L. A. Positional differences in the wound transcriptome of skin and oral mucosa. BMC Genomics 2010, 11, 1-15.
32. Yaron, J. R.; Chen, H.; Ambadapadi, S.; Zhang, L.; Tafoya, A. M.; Munk, B. H.; Wakefield, D. N.; Fuentes, J.; Marques, B. J.; Harripersaud, K.; et al. Serp-2, a virus-derived apoptosis and inflammasome inhibitor, attenuates liver ischemia-reperfusion injury in mice. J. Inflamm. (United Kingdom) 2019, 16.
33. Tardif, J.-C.; L′Allier, P. L.; Grégoire, J.; Ibrahim, R.; McFadden, G.; Kostuk, W.; Knudtson, M.; Labinaz, M.; Waksman, R.; Pepine, C. J.; et al. A randomized controlled, phase 2 trial of the viral serpin Serp-1 in patients with acute coronary syndromes undergoing percutaneous coronary intervention. Circ. Cardiovasc. Interv. 2010, 3, 543-8.
34. Zhang, L.; Yaron, J. R.; Tafoya, A. M.; Wallace, S. E.; Kilbourne, J.; Haydel, S.; Rege, K.; McFadden, G.; Lucas, A. R. A Virus-Derived Immune Modulating Serpin Accelerates Wound Closure with Improved Collagen Remodeling. J. Clin. Med. 2019, 8, 1626.
35. Alexander, H.; Brown, S.; Danby, S.; Flohr, C. Research Techniques Made Simple; Transepidermal Water Loss Measurement as a Research Tool. J. Invest. Dermatol. 2018, 138, 2295-2300.el.
36. Rettinger, C. L.; Fletcher, J. L.; Carlsson, A. H.; Chan, R. K. Accelerated epithelialization and improved wound healing metrics in porcine full-thickness wounds transplanted with full-thickness skin micrografts. Wound Repair Regen. 2017, 25, 816-827.
37. Borowiec, A. M.; Sydora, B. C.; Doyle, J.; Guan, L. L.; Churchill, T. A.; Madsen, K.; Fedorak, R. N. Small bowel fibrosis and systemic inflammatory response after ileocolonic anastomosis in IL-10 null mice. J. Surg. Res. 2012, 178, 147-154.
38. Ghosh, D.; Godeshala, S.; Nitiyanandan, R.; Islam, M. S.; Yaron, J. R.; Dicaudo, D.; Kilbourne, J.; Rege, K. Copper-Eluting Fibers for Enhanced Tissue Sealing and Repair. ACS Appl. Mater. Interfaces 2020, 12, 27951-27960.
39. Fang, R. C.; Kryger, Z. B.; Buck, D. W.; De La Garza, M.; Galiano, R. D.; Mustoe, T. A. Limitations of the db/db mouse in translational wound healing research; Is the NONcNZO10 polygenic mouse model superior? Wound Repair Regen. 2010, 18, 605-613.
40. Mamat, U.; Wilke, K.; Bramhill, D.; Schromm, A. B.; Lindner, B.; Kohl, T. A.; Corchero, J. L.; Villaverde, A.; Schaffer, L.; Head, S. R.; et al. Detoxifying Escherichia coli for endotoxin-free production of recombinant proteins. Microb. Cell Fact. 2015, 14, 1-15.
41. Yaron, J. R.; Ambadapadi, S.; Zhang, L.; Lucas, A. Kinetic Measurement of Serpin Inhibitory Activity by Real-Time Fluorogenic Biochemical Assay. Methods Mol. Biol. 2018, 1826, 65-71.

### Example 4—SERPINB3A Mediates Wound Healing and Tissue Regeneration

Purpose: We will elucidate a new mechanism of regulation for partial epidermal EMT in wound healing mediated by Serpinb3a.

Methodology: NCBI Gene Expression Omnibus (GEO) was queried and GEO2R was used to evaluate gene expression levels of Serpinb3a in acute and diabetic wounds. Immunoblot for Serpinb3a and immunohistochemistry for E-cadherin and Slug was performed on healing skin of immunocompetent Balb/c mice with 5-mm full-thickness splinted wounds at 6 hrs and 2, 4 and 7 days post-wounding. Full-length Serpinb3a was expressed in E. coli and purified to endotoxin-free levels. Serpinb3a identity and inhibitory function was characterized by immunoblot and Cathepsin G assay. Therapeutic efficacy of Serpinb3a was evaluated in 5-mm full-thickness splinted wounds in C57BL6/J and diabetic and obese BKS.Cg-Dock7^{m}+/+Lepr^{db}/J (db/db) mice. Wound closure was evaluated by planimetry. In vitro closure was measured by HaCaT scratch assay and compared to EGF.

Non-Limiting, Exemplary Results: NCBI GEO analysis indicated skin-specific expression of Serpinb3a after dermal wounding, but not mucosal (tongue) wounding (p<0.001, N=3/group), and further indicated that Serpinb3a expression was lower in migrating keratinocytes at the wound edge of db/db but not C57BL6/J mice (p<0.05, N=3/group). Immunoblot analysis of wounds in Balb/c mice demonstrated high levels of Serpinb3a within 6 hours of wounding, peaking at 2-4 days and resolving by day 7 post-wounding (N=3/timepoint). Protein levels of Serpinb3a correlated tightly with immunohistochemical markers of EMT (E-cadherin loss and nuclear localization of Slug) in wound edge keratinocytes. Recombinant Serpinb3a was topically applied to acute and diabetic wounds. Significant acceleration of early closure, during the epidermal EMT phase of healing, was observed with exogenous Serpinb3a in both C57BL6/J and db/db mice (p<0.05, N=2-8). Treatment of HaCaT cells with Serpinb3a significantly accelerated wound closure compared to controls and at levels comparable to EGF (p<0.01, N=2 independent experiments).

Conclusion: Serpinb3a is a new endogenous mediator of wound healing in the skin and represents a therapeutic modulator to accelerate poorly healing wounds. Topical or controlled delivery of Serpinb3a can provide a new treatment approach for tissue repair and regeneration.

### Example 5

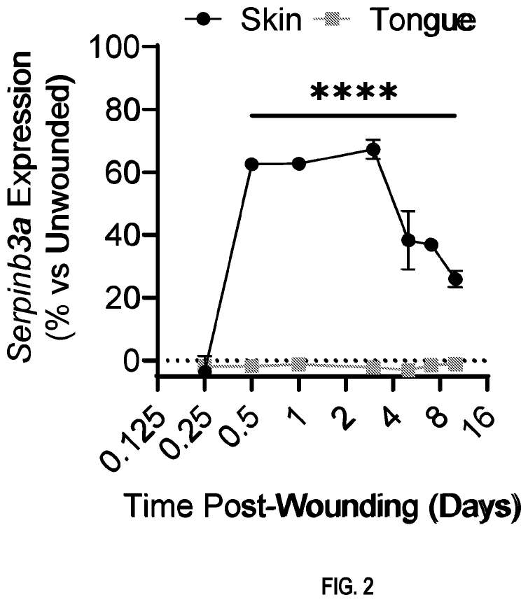

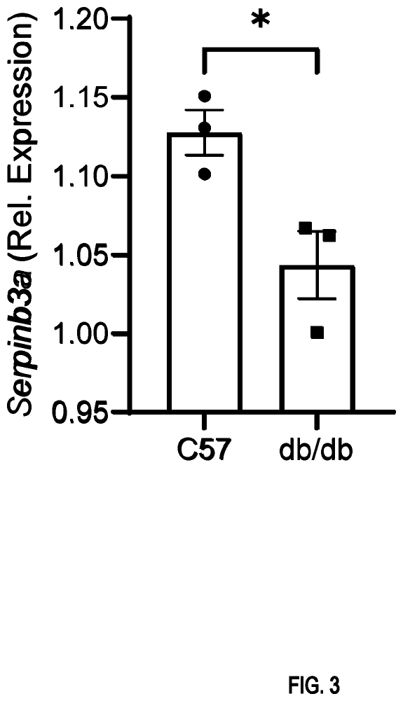

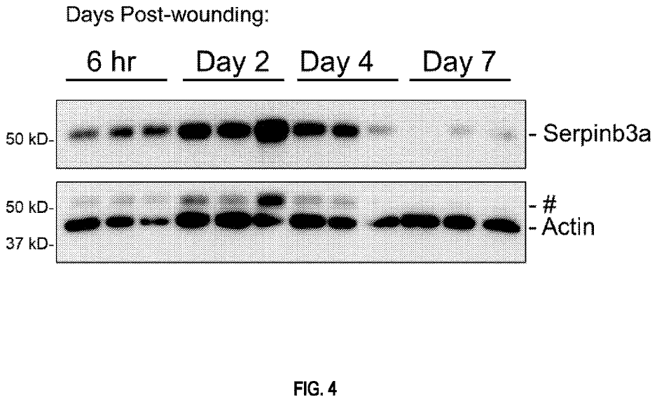

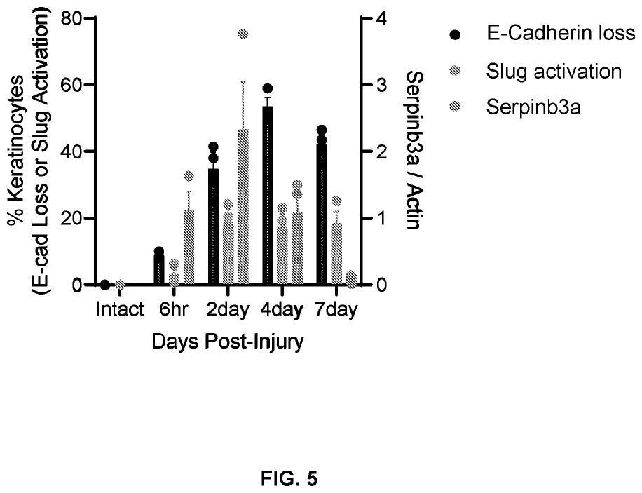

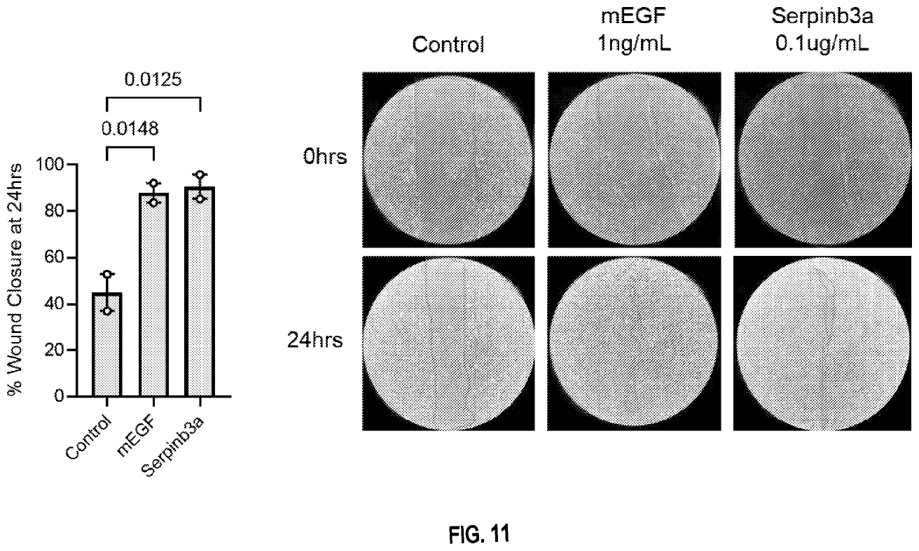

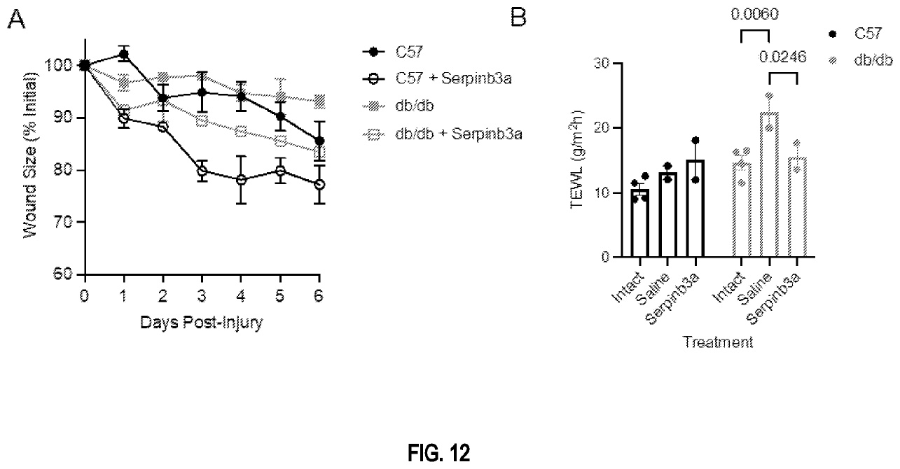

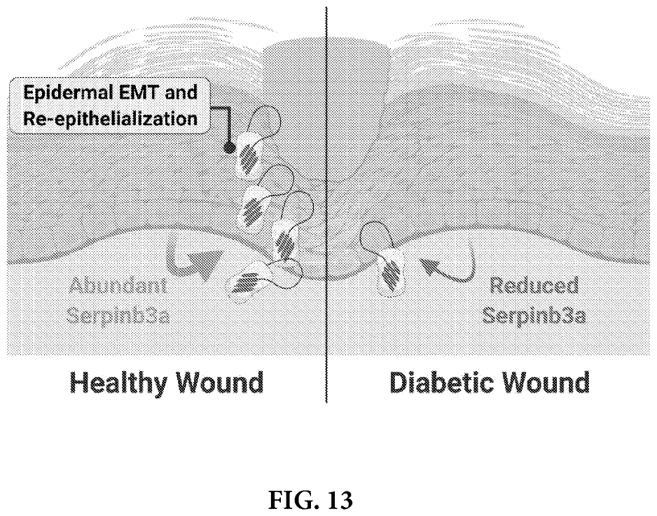

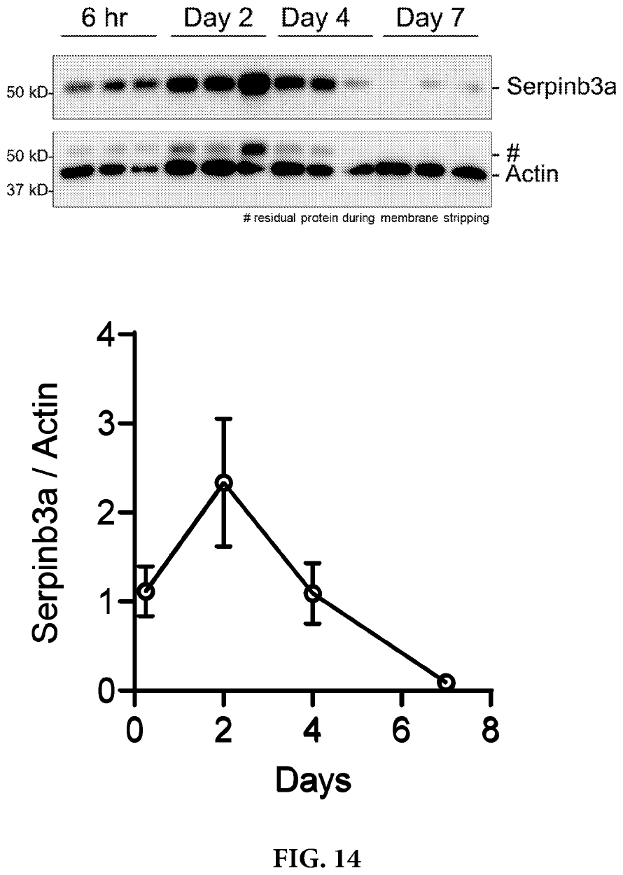

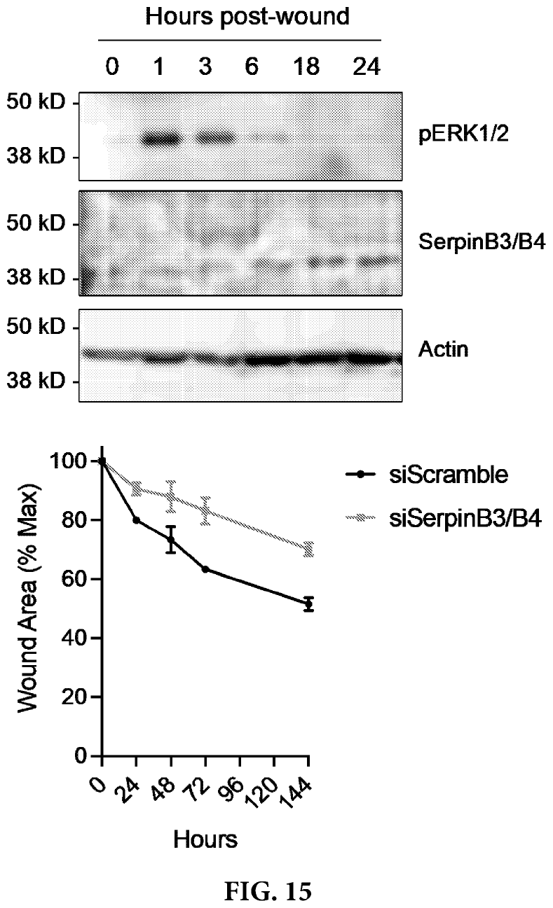

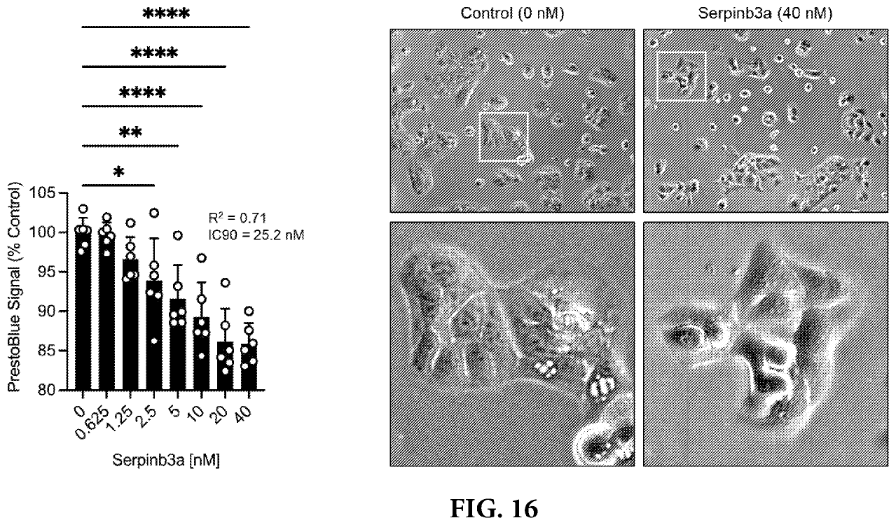

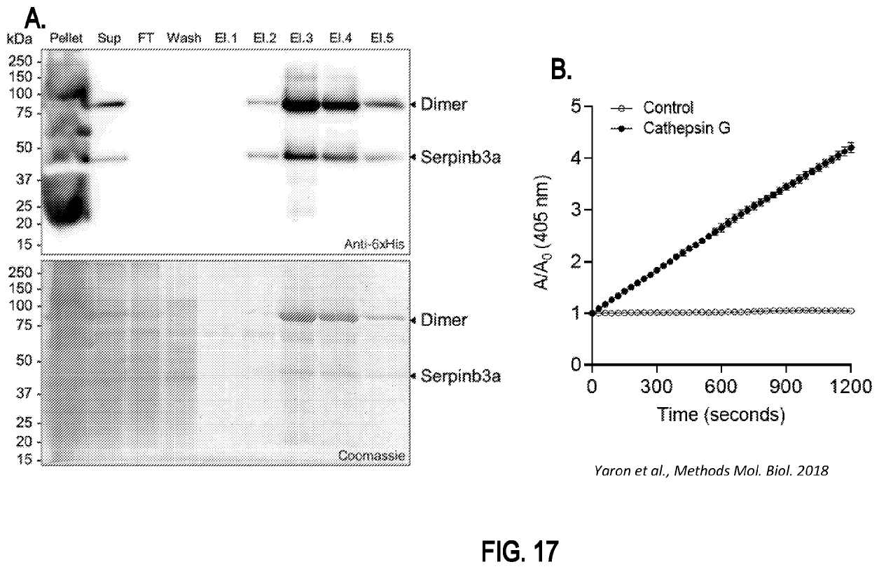

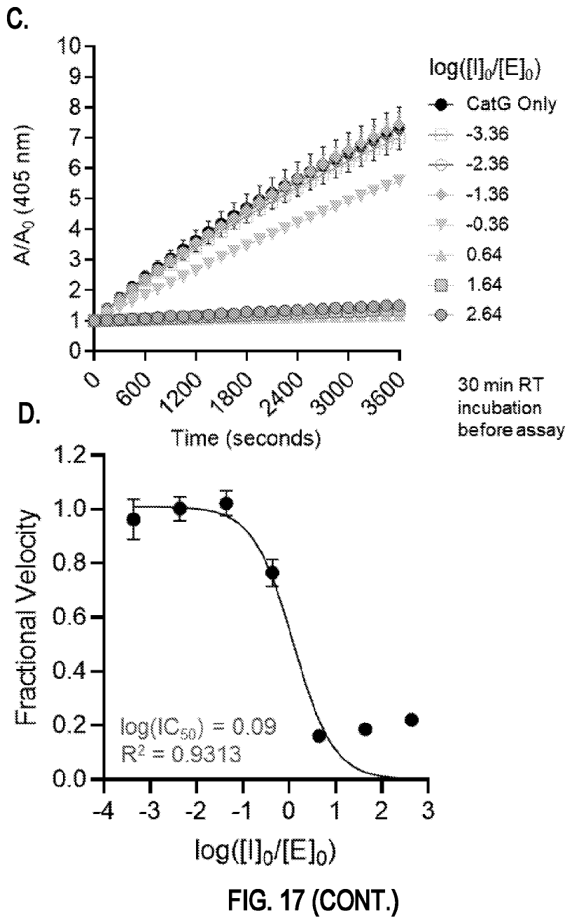

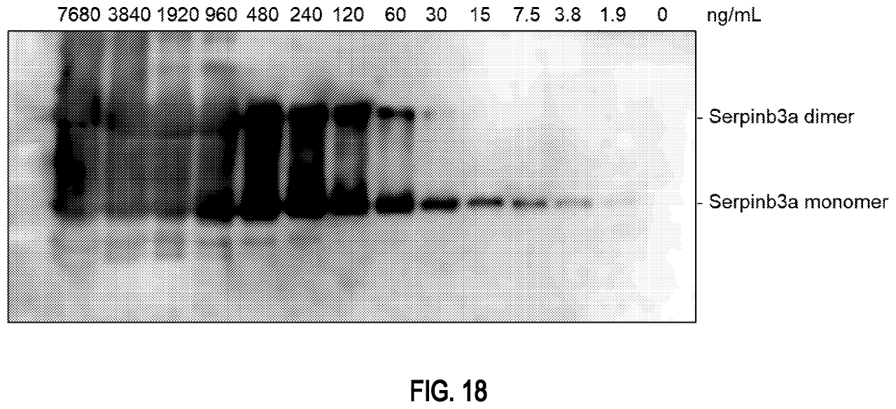

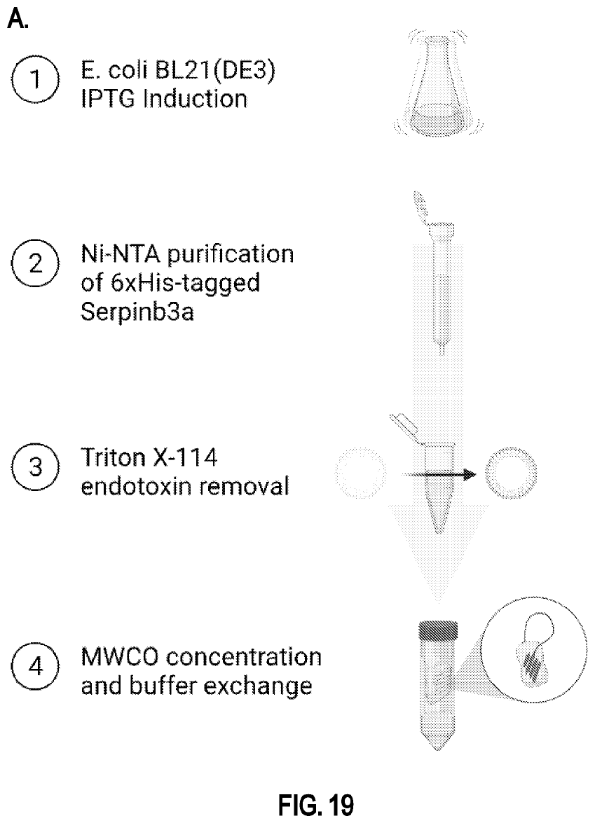

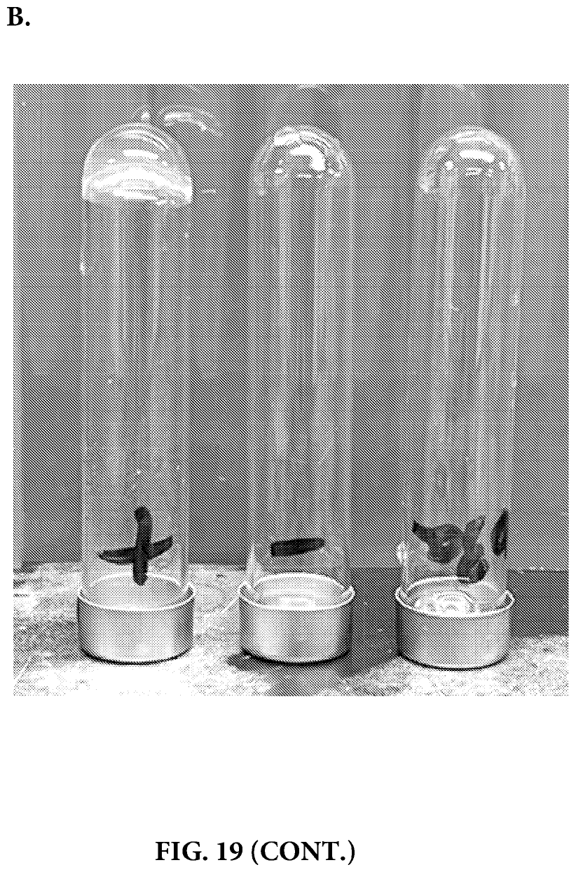

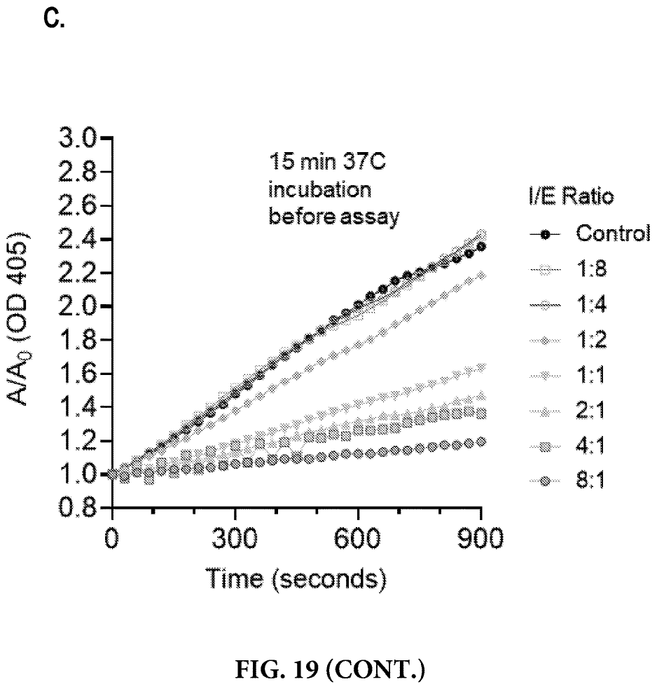

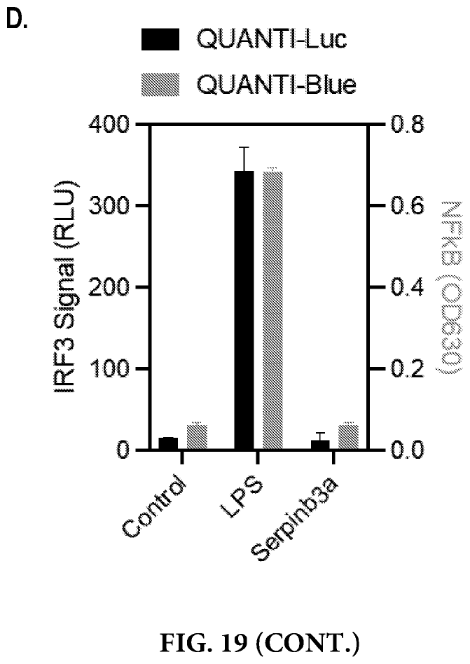

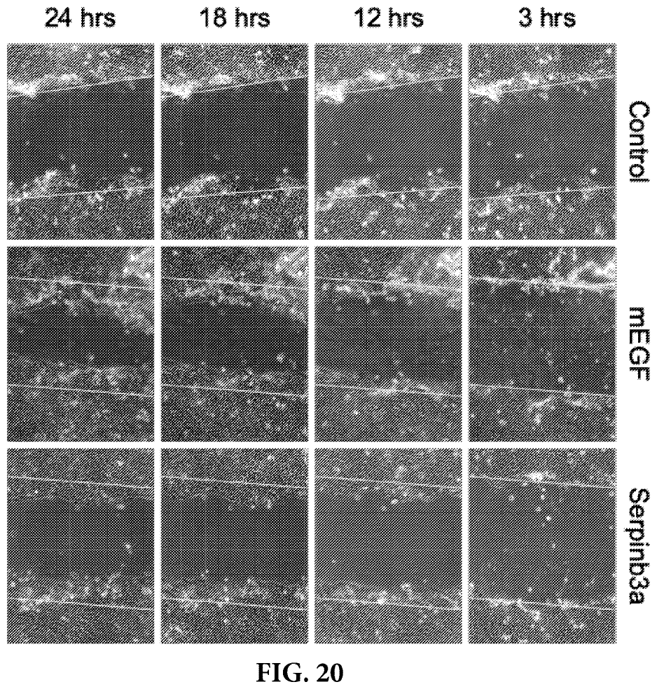

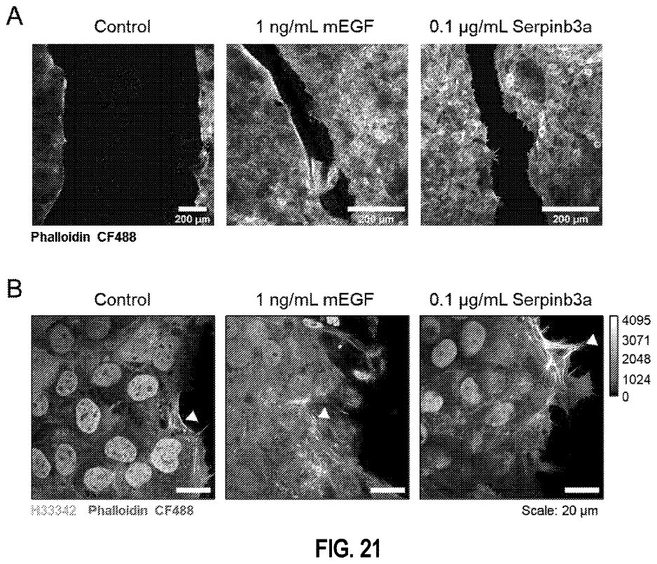

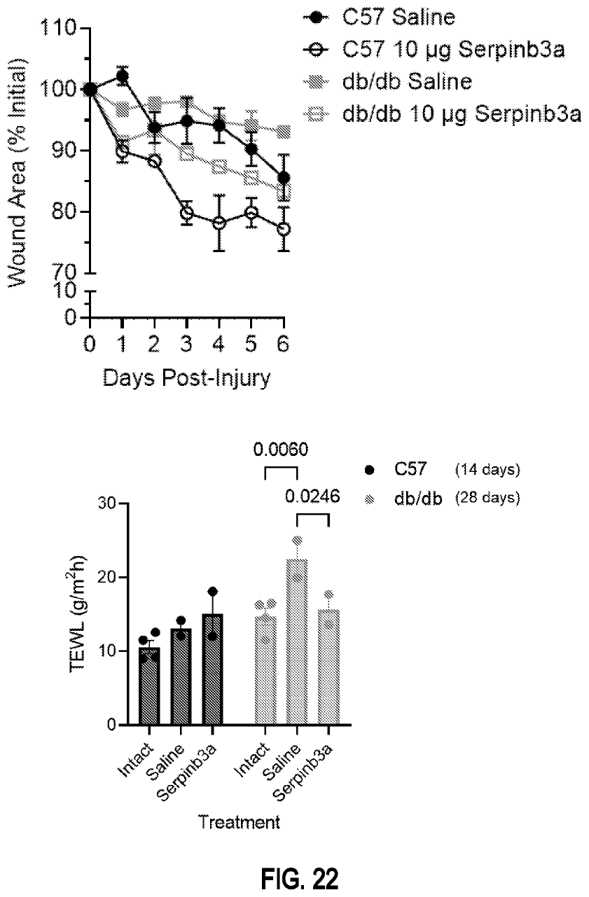

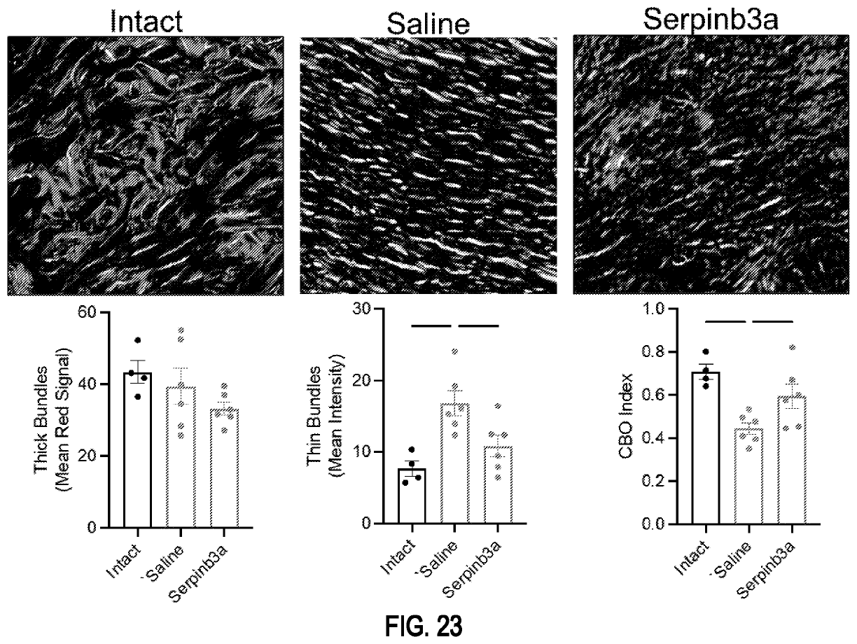

Public database mining: Serpinb3a dynamics in wound healing (FIGS. 2 and 3)
Public dataset mining indicates a skin-specific role for Serpinb3a in healing
Serpinb3a is upregulated only in epidermal wounds, but not in mucosal wounds (FIG. 2).
Serpinb3a is downregulated in migrating peri-wound keratinocytes from db/db mice (FIG. 3).
Without wishing to be bound by theory, Serpinb3a plays a mechanistic role in wound healing (FIG. 13).
Serpinb3a is rapidly and robustly expressed in early wound healing (FIG. 14).
Protein levels of Serpinb3a rapidly respond to skin injury.
Serpinb3a expression correlates with markers of EMT in acute wound healing in mice (FIG. 5). Serpinb3a kinetics correlate with the epidermal EMT window. Histologic markers for EMT were collected from acute wounds in Balb/c mice at the indicated time point. Namely, the loss of E-cadherin signal and the presence of the Slug transcription factor in Keratinocytes at the edge of the wounds. Levels were found to correlate with expression of Serpinb3a measured by Western blot analysis.
Human keratinocytes express SerpinB3/B4 after wounding in vitro and knockdown slows closure (FIG. 15).
Extracellular Serpinb3a slows cell proliferation and promotes EMT-like morphology (FIG. 16). Serpinb3a slows proliferation and promotes morphologic changes.
Production and validation of recombinant Serpinb3a (FIG. 17)
Serpinb3a (B3/B4) are glycosylated and have no secretion signal=difficult to scale and control for therapeutic development. Functional Serpinb3a can be produced abundantly in bacteria.
Protein polishing of Serpinb3a (FIG. 19)
Endotoxin-free preparations are function and non-inflammatory.
FIG. 19 panel B: <0.02 EU/μg protein: FDA Limit: 5 EU/kg bodyweight/day; Eq. mouse: 0.1 EU/20g mouse/day. Can administer at least 5 μg protein per day and remain within FDA guidelines.
FIG. 11 and FIG. 20: Serpinb3a enhances wound closure in vitro. Exogenous Serpinb3a accelerates human keratinocyte scratch closure.
Serpinb3a promotes actin remodeling at the wound front in vitro. Serpinb3a induces cytoskeletal responses during scratch closure. (FIGS. 21 and 22)
Early Serpinb3a treatment enhances ECM remodeling after wound closure. Topical Serpinb3a modulates early wound closure and healing quality in mice. (FIG. 23)

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A polypeptide comprising an amino acid sequence at least 80% identical to an amino acid sequence selected from:
a.
(SEQ ID NO: 18)
MHLFAEATTKFTLELYRQLRESDNNIFYSPISMMTALAMLQLGAKGNTEK
QIEKVLQFNETTKKTTEKSAHCHDEENVHEQFQKLMTQLNKSNDAYDLK
AANSIYGAKGFPFVQTFLEDIKEYYQANVESLDFEHAAEESEKKINSWVE
SQTNGKIKDLFPNGSLNRSTIMVLVNAVYFKGQWNHKFDEKHTTEEKFWL
NKNTSKPVQMMKQNIEFNFMFLEDVQAKIVEIPYKGKELSMIVLLPVEIN
GLKQLEEQLTADKLLEWTRAENMHMTELYLSLPRFKVDEKYDLPIPLEH
MGMVDAFDPQKADFSGMSSTQGLVVSKVLHKSFVEVNEEGTEAAAATG
VEVSLTSAQIAEDFCCDHPFLFFIIHRKTNSILFFGRISSP;
b.
(SEQ ID NO: 19)
MNSLSEANTKFMFDLFQQFRKSKENNIFYSPISITSALGMVLLGAKDNTA
QQIKKVLHFDQVTENTTGKAATYHVDRSGNVHHQFQKLLTEFNKSTDAY
ELKIANKLFGEKTYLFLQEYLDAIKKFYQTSVESVDFANAPEESRKKINS
WVESQTNEKIKNLIPEGNIGSNTTLVLVNAIYFKGQWEKKFNKEDTKEEK
FWPNKNTYKSIQMMRQYTSFHFASLEDVQAKVLEIPYKGKDLSMIVLLPN
EIDGLQKLEEKLTAEKLMEWTSLQNMRETRVDLHLPRFKVEESYDLKDT
LRTMGMVDIFNGDADLSGMTGSRGLVLSGVLHKAFVEVTEEGAEAAAA
TAVVGFGSSPTSTNEEFHCNHPFLFFIRQNKTNSILFYGRFSSP;
c.
(SEQ ID NO: 20)
MNSLSEANTKFMFDLFQQFRKSKENNIFYSPISITSALGMVLLGAKDNTA
QQISKVLHFDQVTENTTEKAATYHVDRSGNVHHQFQKLLTEFNKSTDAY
ELKIANKLFGEKTYQFLQEYLDAIKKFYQTSVESTDFANAPEESRKKINS
WVESQTNEKIKNLFPDGTIGNDTTLVLVNAIYFKGQWENKFKKENTKEEK
FWPNKNTYKSVQMMRQYNSFNFALLEDVQAKVLEIPYKGKDLSMIVLLPN
EIDGLQKLEEKLTAEKLMEWTSLQNMRETCVDLHLPRFKMEESYDLKDT
LRTMGMVNIFNGDADLSGMTWSHGLSVSKVLHKAFVEVTEEGVEAAAA
TAVVVVELSSPSTNEEFCCNHPFLFFIRQNKTNSILFYGRFSSP;
or a fragment thereof.

2. The polypeptide of claim 1, wherein the polypeptide is recombinantly produced.

3. The polypeptide of claim 1, wherein the fragment comprises an amino acid sequence at least 80% identical to the amino acid sequence of:
a.
(SEQ ID NO: 8)
GTEAAAATGVEVSLTSAQIA;
b.
(SEQ ID NO: 9)
GAEAAAATAVVGFGSSPTST;
or
c.
(SEQ ID NO: 10)
GVEAAAATAVVVVELSSPST.

4. The polypeptide of claim 1, wherein the fragment comprises a truncation mutant.

5. The polypeptide of claim 4, wherein the truncation mutant comprises an N-truncated mutant or a C-truncated mutant.

6. The polypeptide of claim 4, wherein the truncation mutant comprises a reactive center loop truncation mutant.

7. The polypeptide of claim 1, wherein
a) positions 71-76 of the polypeptide according to SEQ ID NO: 5 are replaced with TYHVDRS (SEQ ID NO: 11) or YHVDRS (SEQ ID NO: 12), wherein positions 346-356 according to SEQ ID NO: 5 are replaced with VVGFGSSPTS (SEQ ID NO: 13) or VVVVELSSPS (SEQ ID NO: 14), or any combination thereof;
b) positions 72-78 of the polypeptide according to SEQ ID NO: 6 are replaced with HCHDEE (SEQ ID NO: 15) or YHVDRS (SEQ ID NO: 12), wherein positions 349-358 according to SEQ ID NO: 6 are replaced with VEVSLTSAQIA (SEQ ID NO: 16) or VVVVELSSPS (SEQ ID NO: 14), or any combination thereof;
c) positions 73-78 of the polypeptide according to SEQ ID NO: 7 are replaced with HCHDEE (SEQ ID NO: 15) or TYHVDRS (SEQ ID NO: 11), wherein positions 349-358 according to SEQ ID NO: 7 are replaced with VEVSLTSAQIA (SEQ ID NO: 16) or VVGFGSSPTS (SEQ ID NO: 13), or any combination thereof.

8. The polypeptide of claim 1, wherein the polypeptide comprises one or more post-translational modifications.

9. The polypeptide of claim 8, wherein the post-translational modification is selected from the group consisting of PEGylation, sialylation, glycosylation, acetylation, acylation, lipid modification, palmitoylation, palmitate addition, phosphorylation, Fc-Ig fusion, and glycolipid modification.

10. The polypeptide of claim 1, wherein the polypeptide is degylcosylated.

11. The polypeptide of claim 1, wherein the polypeptide comprises at least one insertion, deletion, or mutation.

12. A chimeric polypeptide or fragment thereof, wherein the chimeric polypeptide comprises an amino acid sequence at least 80% identical to an amino acid sequence selected from:
a. MHLFAEATTKFTLELYRQLRESDNNIFYSPISMMTALAMLQLGAKGNTEKQI EKVLQFNETTKKTTEKSAHCHDEENVHEQFQKLMTQLNKSNDAYDLKAANS IYGAKGFPFVQTFLEDIKEYYQANVESLDFEHAAEESEKKINSWVESQTNGKI KDLFPNGSLNRSTIMVLVNAVYFKGQWNHKFDEKHTTEEKFWLNKNTSKPV QMMKQNIEFNFMFLEDVQAKIVEIPYKGKELSMIVLLPVEINGLKQLEEQLTA DKLLEWTRAENMHMTELYLSLPRFKVDEKYDLPIPLEHMGMVDAFDPQKAD FSGMSSTQGLVVSKVLHKSFVEVNEEGTEAAAATGVEVSLTSAQIAEDFCCD HPFLFFIIHRKTNSILFFGRISSP (SEQ ID NO: 18), wherein positions 71-76 are replaced with TYHVDRS (SEQ ID NO: 11) or YHVDRS (SEQ ID NO: 12), wherein positions 346-356 are replaced with VVGFGSSPTS (SEQ ID NO: 13) or VVVVELSSPS (SEQ ID NO: 14), or any combination thereof;;
b. MNSLSEANTKFMFDLFQQFRKSKENNIFYSPISITSALGMVLLGAKDNTAQQI KKVLHFDQVTENTTGKAATYHVDRSGNVHHQFQKLLTEFNKSTDAYELKIA NKLFGEKTYLFLQEYLDAIKKFYQTSVESVDFANAPEESRKKINSWVESQTNE KIKNLIPEGNIGSNTTLVL VNAIYFKGQWEKKENKEDTKEEKFWPNKNTYKSI QMMRQYTSFHFASLEDVQAKVLEIPYKGKDLSMIVLLPNEIDGLQKLEEKLT AEKLMEWTSLQNMRETRVDLHLPRFKVEESYDLKDTLRTMGMVDIFNGDA DLSGMTGSRGLVLSGVLHKAFVEVTEEGAEAAAATAVVGFGSSPTSTNEEFH CNHPFLFFIRQNKTNSILFYGRFSSP (SEQ ID NO: 19), wherein positions 72-78 are replaced with HCHDEE (SEQ ID NO: 15) or YHVDRS (SEQ ID NO: 12), wherein positions 349-358 are replaced with VEVSLTSAQIA (SEQ ID NO: 16) or VVVVELSSPS (SEQ ID NO: 14), or any combination thereof, or
c. MNSLSEANTKFMFDLFQQFRKSKENNIFYSPISITSALGMVLLGAKDNTAQQI SKVLHFDQVTENTTEKAATYHVDRSGNVHHQFQKLLTEFNKSTDAYELKIA NKLFGEKTYQFLQEYLDAIKKFYQTSVESTDFANAPEESRKKINSWVESQTNE KIKNLFPDGTIGNDTTLVL VNAIYFKGQWENKFKKENTKEEKFWPNKNTYKS VQMMRQYNSFNFALLEDVQAKVLEIPYKGKDLSMIVLLPNEIDGLQKLEEKL TAEKLMEWTSLQNMRETCVDLHLPRFKMEESYDLKDTLRTMGMVNIFNGD ADLSGMTWSHGLSVSKVLHKAFVEVTEEGVEAAAATAVVVVELSSPSTNEE FCCNHPFLFFIRQNKTNSILFYGRFSSP (SEQ ID NO: 20), wherein positions 73-78 of the polypeptide are replaced with HCHDEE (SEQ ID NO: 15) or TYHVDRS (SEQ ID NO: 11), wherein positions 349-358 are replaced with VEVSLTSAQIA (SEQ ID NO: 16) or VVGFGSSPTS (SEQ ID NO: 13), or any combination thereof.

13. The polypeptide of claim 12, wherein the polypeptide comprises one or more post-translational modifications.

14. The polypeptide of claim 13, wherein the post-translational modification is selected from the group consisting of PEGylation, sialylation, glycosylation, acetylation, acylation, lipid modification, palmitoylation, palmitate addition, phosphorylation, Fc-Ig fusion, and glycolipid modification.

15. The polypeptide of claim 12, wherein the polypeptide is degylcosylated.

16. The polypeptide of claim 12, wherein the polypeptide comprises at least one insertion, deletion, or mutation.

17.-33. (canceled)


SEQUENCE LISTING (22 sequences)

SEQ ID NO: 1 - protein, 387 aa, Mus musculus
MHLFAEATTKFTLELYRQLRESDNNIFYSPISMMTALAMLQLGAKGNTEKQIEKVLQFNETTKKTTEKSAHCHDEENVHEQFQKLMTQLNKSNDAYDLKAANSIYGAKGFPFVQTFLEDIKEYYQANVESLDFEHAAEESEKKINSWVESQTNGKIKDLFPNGSLNRSTIMVLVNAVYFKGQWNHKFDEKHTTEEKFWLNKNTSKPVQMMKQNIEFNFMFLEDVQAKIVEIPYKGKELSMIVLLPVEINGLKQLEEQLTADKLLEWTRAENMHMTELYLSLPRFKVDEKYDLPIPLEHMGMVDAFDPQKADFSGMSSTQGLVVSKVLHKSFVEVNEEGTEAAAATGVEVSLTSAQIAEDFCCDHPFLFFIIHRKTNSILFFGRISSP

SEQ ID NO: 2 - protein, 390 aa, Homo sapiens
MNSLSEANTKFMFDLFQQFRKSKENNIFYSPISITSALGMVLLGAKDNTAQQIKKVLHFDQVTENTTGKAATYHVDRSGNVHHQFQKLLTEFNKSTDAYELKIANKLFGEKTYLFLQEYLDAIKKFYQTSVESVDFANAPEESRKKINSWVESQTNEKIKNLIPEGNIGSNTTLVLVNAIYFKGQWEKKFNKEDTKEEKFWPNKNTYKSIQMMRQYTSFHFASLEDVQAKVLEIPYKGKDLSMIVLLPNEIDGLQKLEEKLTAEKLMEWTSLQNMRETRVDLHLPRFKVEESYDLKDTLRTMGMVDIFNGDADLSGMTGSRGLVLSGVLHKAFVEVTEEGAEAAAATAVVGFGSSPTSTNEEFHCNHPFLFFIRQNKTNSILFYGRFSSP

SEQ ID NO: 3 - protein, 390 aa, Homo sapiens
MNSLSEANTKFMFDLFQQFRKSKENNIFYSPISITSALGMVLLGAKDNTAQQISKVLHFDQVTENTTEKAATYHVDRSGNVHHQFQKLLTEFNKSTDAYELKIANKLFGEKTYQFLQEYLDAIKKFYQTSVESTDFANAPEESRKKINSWVESQTNEKIKNLFPDGTIGNDTTLVLVNAIYFKGQWENKFKKENTKEEKFWPNKNTYKSVQMMRQYNSFNFALLEDVQAKVLEIPYKGKDLSMIVLLPNEIDGLQKLEEKLTAEKLMEWTSLQNMRETCVDLHLPRFKMEESYDLKDTLRTMGMVNIFNGDADLSGMTWSHGLSVSKVLHKAFVEVTEEGVEAAAATAVVVVELSSPSTNEEFCCNHPFLFFIRQNKTNSILFYGRFSSP

SEQ ID NO: 4 - protein, 391 aa, synthetic construct
  VARIANT 2: note = H or N
  VARIANT 3: note = L or S
  VARIANT 4: note = F or L
  VARIANT 5: note = A or S
  VARIANT 8: note = T or N
  VARIANT 12: note = T or M
  VARIANT 13: note = L or F
  VARIANT 14: note = E or D
  VARIANT 16: note = Y or F
  VARIANT 17: note = R or Q
  VARIANT 19: note = L or F
  VARIANT 21: note = E or K
  VARIANT 23: note = may be deleted
  VARIANT 24: note = D or E
  VARIANT 34: note = M or I
  VARIANT 35: note = M or T
  VARIANT 36: note = T or S
  VARIANT 39: note = A or G
  VARIANT 41: note = L or V
  VARIANT 42: note = Q or L
  VARIANT 47: note = G or D
  VARIANT 50: note = E or A
  VARIANT 51: note = K or Q
  VARIANT 54: note = E, K or S
  VARIANT 58: note = Q or H
  VARIANT 62: note = T or V
  VARIANT 64: note = K or E
  VARIANT 65: note = K or N
  VARIANT 68: note = E or G
  VARIANT 70: note = S or A
  VARIANT 72: note = H or T
  VARIANT 73: note = may be deleted
  VARIANT 74: note = may be deleted
  VARIANT 75: note = C or V
  VARIANT 76: note = H or D
  VARIANT 77: note = D or R
  VARIANT 78: note = E or S
  VARIANT 79: note = E or G
  VARIANT 83: note = E or H
  VARIANT 89: note = M or L
  VARIANT 92: note = L or F
  VARIANT 96: note = N or T
  VARIANT 100: note = D or E
  VARIANT 103: note = A or I
  VARIANT 106: note = S or K
  VARIANT 108: note = Y or F
  VARIANT 110: note = A or E
  VARIANT 112: note = G or T
  VARIANT 113: note = F or Y
  VARIANT 114: note = P, L or Q
  VARIANT 116: note = V or L
  VARIANT 118: note = T or E
  VARIANT 119: note = F or Y
  VARIANT 121: note = E or D
  VARIANT 122: note = D or A
  VARIANT 125: note = E or K
  VARIANT 126: note = Y or F
  VARIANT 129: note = A or T
  VARIANT 130: note = N or S
  VARIANT 134: note = L, V or T
  VARIANT 137: note = E or A
  VARIANT 138: note = H or N
  VARIANT 140: note = A or P
  VARIANT 144: note = E or R
  VARIANT 157: note = G or E
  VARIANT 163: note = F or I
  VARIANT 165: note = N, E or D
  VARIANT 167: note = S, N or T
  VARIANT 169: note = N or G
  VARIANT 170: note = R, S or N
  VARIANT 171: note = S, N or D
  VARIANT 173: note = I or T
  VARIANT 174: note = M or L
  VARIANT 180: note = V or I
  VARIANT 187: note = N or E
  VARIANT 188: note = H, K or N
  VARIANT 191: note = D, N or K
  VARIANT 192: note = E or K
  VARIANT 193: note = K or E
  VARIANT 194: note = H, D or N
  VARIANT 196: note = T or K
  VARIANT 202: note = L or P
  VARIANT 207: note = S or Y
  VARIANT 209: note = P or S
  VARIANT 210: note = V or I
  VARIANT 214: note = K or R
  VARIANT 216: note = N or Y
  VARIANT 217: note = I, T or N
  VARIANT 218: note = E or S
  VARIANT 220: note = N or H
  VARIANT 222: note = M or A
  VARIANT 223: note = F, S or L
  VARIANT 231: note = I or V
  VARIANT 232: note = V or L
  VARIANT 240: note = E or D
  VARIANT 249: note = V or N
  VARIANT 255: note = K or Q
  VARIANT 256: note = Q or K
  VARIANT 260: note = Q or K
  VARIANT 264: note = D or E
  VARIANT 267: note = L or M
  VARIANT 271: note = R or S
  VARIANT 272: note = A or L
  VARIANT 276: note = H or R
  VARIANT 277: note = M or E
  VARIANT 279: note = E, R or C
  VARIANT 280: note = L or V
  VARIANT 281: note = Y or D
  VARIANT 283: note = S or H
  VARIANT 289: note = V or M
  VARIANT 290: note = D or E
  VARIANT 292: note = K or S
  VARIANT 296: note = P or K
  VARIANT 297: note = I or D
  VARIANT 298: note = P or T
  VARIANT 300: note = E or R
  VARIANT 301: note = H or T
  VARIANT 307: note = A or I
  VARIANT 310: note = P or G
  VARIANT 311: note = may be deleted
  VARIANT 312: note = K or D
  VARIANT 315: note = F or L
  VARIANT 319: note = S or T
  VARIANT 320: note = S, G or W
  VARIANT 321: note = T or S
  VARIANT 322: note = Q, R or H
  VARIANT 325: note = V or S
  VARIANT 326: note = V or L
  VARIANT 328: note = K or G
  VARIANT 333: note = S or A
  VARIANT 338: note = N or T
  VARIANT 342: note = T, A or V
  VARIANT 349: note = G or A
  VARIANT 350: note = may be deleted
  VARIANT 352: note = E, G or V
  VARIANT 353: note = V or F
  VARIANT 354: note = S, G or E
  VARIANT 355: note = L or S
  VARIANT 356: note = T or S
  VARIANT 357: note = S or P
  VARIANT 358: note = A, T or P
  VARIANT 359: note = Q or S
  VARIANT 360: note = I or T
  VARIANT 361: note = A or N
  VARIANT 363: note = D or E
  VARIANT 365: note = C or H
  VARIANT 375: note = I or R
  VARIANT 376: note = H or Q
  VARIANT 377: note = R or N
  VARIANT 385: note = F or Y
  VARIANT 388: note = I or F
MXXXXEAXTKFXXXLXXQXRXSKXNNIFYSPISXXXALXMXXLGAKXNTXXQIXKVLXFBZXTXXTTXKXAXYHXXXXXNVHXQFQKLXTZXNKSXDAYXLKXANXJXGXKXXXFXQXXLXXIKXXYQXXVESXDFXXAXEESXKKINSWVESQTNXKIKBLXPXGXJXXXTXXVLVNAXYFKGQWXXKFXXXXTXEEKFWXNKNTXKXXQMMXQXXXFXFXXLEDVQAKXXEIPYKGKXLSMIVLLPXEIBGLXXLEEXLTAXKLXEWTXXZNMXXTXXXLXLPRFKXXEXYDLXXXLXXMGMVBXFBXQXADXSGMXXXXGLXXSXVLHKXFVEVXEEGXEAAAATXVVXXXXXXXXXXEXFXCBHPFLFFIXXXKTNSILFXGRXSSP

SEQ ID NO: 5 - protein, 387 aa, Mus musculus; identical to SEQ ID NO: 1

SEQ ID NO: 6 - protein, 390 aa, Homo sapiens; identical to SEQ ID NO: 2

SEQ ID NO: 7 - protein, 390 aa, Homo sapiens; identical to SEQ ID NO: 3

SEQ ID NO: 8 - protein, 20 aa, Mus musculus
GTEAAAATGVEVSLTSAQIA

SEQ ID NO: 9 - protein, 20 aa, Homo sapiens
GAEAAAATAVVGFGSSPTST

SEQ ID NO: 10 - protein, 20 aa, Homo sapiens
GVEAAAATAVVVVELSSPST

SEQ ID NO: 11 - protein, 7 aa, synthetic construct
TYHVDRS

SEQ ID NO: 12 - protein, 6 aa, synthetic construct
YHVDRS

SEQ ID NO: 13 - protein, 10 aa, synthetic construct
VVGFGSSPTS

SEQ ID NO: 14 - protein, 10 aa, synthetic construct
VVVVELSSPS

SEQ ID NO: 15 - protein, 6 aa, synthetic construct
HCHDEE

SEQ ID NO: 16 - protein, 11 aa, synthetic construct
VEVSLTSAQIA

SEQ ID NO: 17 - protein, 6 aa, synthetic construct
HHHHHH

SEQ ID NO: 18 - protein, 387 aa, synthetic construct; identical to SEQ ID NO: 1

SEQ ID NO: 19 - protein, 390 aa, synthetic construct; identical to SEQ ID NO: 2

SEQ ID NO: 20 - protein, 390 aa, synthetic construct; identical to SEQ ID NO: 3

SEQ ID NO: 21 - protein, 7 aa, synthetic construct
  VARIANT 2: note = any residue
  VARIANT 5: note = any large/medium hydrophobic residue
  VARIANT 7: note = any small hydrophobic or polar residue
EXLYXQX

SEQ ID NO: 22 - protein, 4 aa, synthetic construct
  MOD_RES 1: note = N-succinyl-Ala
  MOD_RES 4: note = p-nitroanilide-Phe
AAPF